(19) United States
(12) Patent Application Publication
(10) Pub. No.: US 2007/0275024 A1
(43) Pub. Date: Nov. 29, 2007

(54) Starch Films and Uses Thereof

(75) Inventors: Clifford Leonard Hedley, Norfolk (GB); Tatiana Yakovlevna Bogracheva, Norfolk (GB); Ian Osmond Topliff, Norfolk (GB)

Correspondence Address: MICHAEL BEST & FRIEDRICH LLP, ONE SOUTH PINCKNEY STREET, P O BOX 1806, MADISON, WI, 53701, US

(73) Assignee: PLANT BIOSCIENCE LIMITED, Norwich Norfolk (GB)

(21) Appl. No.: 11/569,801

(22) PCT Filed: Jun. 1, 2005
(86) PCT No.: PCT/GB05/02169

§ 371 Date: Dec. 14, 2006

(30) Foreign Application Priority Data
Jun. 1, 2004 (GB) 0412201.6

(51) Int. Cl. C08L 3/02; A61K 31/718; A61K 39/395; A61K 9/70

(52) U.S. Cl. 424/405; 424/443; 514/60; 536/102

(57) ABSTRACT

The invention provides novel starch films and processes for production thereof based on starch from C-type starch granules (e.g. from legumes) and polyols (e.g. glycerol). In other aspects the invention provides wound dressings and systems for controlling microorganisms using starch films.

### TECHNICAL FIELD

The present invention relates generally to methods and materials for use in preparing and using starch films having improved properties.

### BACKGROUND ART

It is known that when a starch granule suspension is heated (or superheated, i.e. a heat and pressure combination) at a certain temperature the starch supramolecular structure will be disrupted, the granules swell and the amylose from the granules is partially solubilised, to form a starch paste. When the paste is cooled down and the excess water is allowed to evaporate the starch polymers retrograde, forming supramolecular structures (helices, double helices, crystallites). It is assumed these structures form a cross-linked network which is the basis for the film itself, and which is largely responsible for its properties.

Publications concerned with the starch films and their properties include Lawton, J. W. “Effect of starch type on the properties of starch containing films”, Carbohydrate Polymers 29 (1996) 203-208; Rindlav-Westling, A. “Structure, mechanical and barrier properties of amylose and amylopectin films”, Carbohydrate Polymers 36 (1998) 217-224.

Plasticisers such as glycerol have been used in the art, and in certain circumstances these have been shown to affect (both positively and negatively) elasticity and tensile strength—see e.g. Myllarinen, P. et al “Effect of glycerol on behaviour of amylose and amylopectin films”, Carbohydrate Polymers 50 (2002) 355-361. For example, U.S. Pat. No. 6,649,188 discusses starch-based films including plasticisers, generally based on chemically modified starches and waxy starches, which are said to be highly flexible and resistant to penetration by water, oil and/or grease. The film-forming compositions discussed therein comprise, on a dry solids basis, 25 to 75 percent by weight of certain starch derivatives and 25 to 75% primary external plasticizer and may employ starch derivatives can be chemically modified starches that range in molecular weight from 100,000 to 2,000,000.

In some cases, starch films have been combined with other polymers e.g. to provide biodegradeable plastic and plastics (see Lawton et al. supra; also for example U.S. Pat. No. 5,009,648 (Starch containing film ostomy pouches).

The use of certain starches and plasticisers is discussed in an abstract (“Properties of pea starch film plasticized by monosaccharides or polyols”—Zhang et al., 2004 IFT Annual Meeting, July 12-16—Las Vegas, Nev., Session 83C, Food Packaging: Role of films, edible coatings, and biopolymers in food packaging). The films discussed therein, which were apparently formed from starch solutions rather than suspensions, employed a ratio of plasticiser to starch calculated as 0.4, 0.8, and 1.5 for glycerol, sorbitol and maltitol respectively. No details of the starch weight per film were given in the abstract, and no comparison of different starch types is disclosed therein.

Thus to date the utilisation of starch films in industry, particularly those not combined with other polymers, has been limited by their properties, or by finding appropriate applications for those properties. Thus it can be seen that novel starch films having improved properties and\or novel applications for starch films, would provide a contribution to the art.

### DISCLOSURE OF THE INVENTION

The present inventors have extensively investigated the relationship between the, molecular, physico-chemical and thermodynamic characteristics associated with starch granular structure and the properties of the films formed therefrom. By studying the formation and characteristics of films from a wide range of starches that differed in their granular structure, the inventors have been able to formulate a hypothesis that links starch structure, film processing, film structure and film properties.

On the basis of these investigations, the present inventors have demonstrated that starch films have combinations of properties (which properties are selected from, for example, mechanical properties; hydrophilicity; low antigenicity; vapour transmission; water adsorption, stability in water, anti-bacterial and so on) which make them particularly suitable for medical applications (for example as wound dressings, or in other contexts where anti-microbial activity is desirable). Indeed, as described herein, these properties can be tailored according to the particular medical application required.

The inventors have further demonstrated that so-called C-type starches (such as those from grain legumes, e.g. pea seeds) may be particularly suitable for film making, as compared with starches more commonly used A-type (mainly from maize and wheat) and B-type (mainly from potato). Films derived from C-type starches are particularly suitable for medical applications. Additionally, when such films are prepared using polyols such as glycerol as a plasticiser, their properties in this respect are further enhanced.

Thus in one aspect of the present invention there is provided a process for producing a starch film comprising the steps of:

(i) providing a preparation of starch from C-type starch granules,

(ii) heating, with agitating, the starch as a suspension in water and a polyol to form a starch paste,

(iii) cooling and drying the starch paste to form a starch film.

It will be appreciated that by “agitating” is meant achieving the required suspension, and the term is used broadly to encompass means for achieving this.

The processes claimed herein differ from that described by Zhang et al., for example in that they employ suspensions, in respect of the amount of starch per unit area, or in the ratio of plasticiser to starch used in the process. The starch films prepared by Zhang et al. can be described as follows:

| | mM/100 g | | | |
|---|---|---|---|---|
| | of | Plasticizer | | Plasticizer/Starch |
| Plasticizer | solution | Concentration % | Starch % | ratio |
| Glycerol | 13.031 | 1.20 | 3 | 0.400 |
| Sorbitol | 13.031 | 2.37 | 3 | 0.791 |
| Maltitol | 13.031 | 4.48 | 3 | 1.494 |

The present inventors have found that for plasticisers such as glycerol which are liquid at room temperature, preferred films are those in which the ratio is greater than or equal to 0.5. Conversely for plasticisers such as sorbitol and maltitol which are solid at room temperature, such ratios give films with poor properties after storage (see Table 5) and therefore it is preferred to use a ratio less than 0.5. As used herein “room temperature” is 20° C.

Thus in the processes of the present aspect where the plasticiser is sorbitol or maltitol, the ratio may be less than 0.5, and where it is glycerol, the ratio may be greater than or equal to 0.5.

C-Type Starch Granules

Those skilled in the art are familiar with starch granules having C-type crystallinity, which can be determined for example using polarized light in conjunction with differential scanning calorimetry or x-ray diffraction. The crystal structure of starches and starch granules has been reviewed in a number of papers; see for example Perez et al. “Structural features of starch”, Carbohydrates in Europe 15 (October 1996) which discusses the 2 types of starch polymorph—A and B. Briefly, A-type starch contains the former, while B-type starch contains the latter. C-type starch, found for example in peas, contains both A- and B-type crystal structures, and was discussed for example by Sarke, A. et al “The crystal structures of A-, B- and C-polymorphs of amylose and starch”, Starch Starke 30 (1978) Nr 3, p73-78 and Zobel, H. F. “Starch crystal transformations and their industrial importance”, Starch Starke 40 (1988) Nr 1, S 1-7. More recently the C-type structure was investigated e.g. in Bogracheva, T. et al “Structural studies of starches with different water contents”, Biopolymers, vol 64, 268-281 (2002); Bogracheva, T. et al “The granular structure of C-type pea starch and its role in gelatinisation”, Biopolymers, vol 45, 323-332 (1998)). Such starches typically also have a relatively high amylose content and this additionally makes them suitable for use in the present invention.

One publication which was concerned with the amylose content of starch films was Forssell, P. et al “Oxygen Permeability of amylose and amylopectin films”, Carbohydrate Polymers, vol 47, issue 2, (February 2002) 125-129. This described the preparation of films from peas. However, significantly, such films were not prepared with glycerol.

Preferred starches for use in the present invention are naturally occurring ones obtainable from plants i.e. are not chemically modified.

Preferred sources of C-type granules are legume seeds. Although not wishing to be bound by any particular theory, it is believed that the C-type crystalline structure (i.e. A & B crystallites in each granule) and architecture (generally B-type crystallites in the centre surrounded by the A-type crystallites) of most legume-derived granules provides particularly advantageous properties when starch films are prepared therefrom in the presence of glycerol. When starch granules from such plants are heated in excess water the crystal structures break down, generally with the B-type crystals melting first.

There are reports in the literature that sweet potato (Ipomoea batatas) may have either C- or A-type starch, possibly dependent on growing temperature (Hizukuri, 1969 “The effect of environment temperature of plants on the physicochemical properties of their starches. 17 (1), 73-88”.

Thus as used herein, the term “C-type starch granule” means a starch granule comprising both A-/B-polymorphs in respective proportions of between 10/90 and 90/10, more preferably between 40/60 and 60/40, for dry starch powders (at 12-14% H₂O), and for wet starch 10/90 and 90/10, 30/70 and 70/30, 35/65 and 65/35, respectively. In each case this is measured using x-ray diffraction, when the starches are equilibrated at 40-60% RH, or using a DSC method when starches are suspended in salt solution, e.g. as in Bogracheva, T. et al “Structural studies of starches with different water contents”, Biopolymers, vol 64, 268-281 (2002).

The amylose content is preferably in the range 5-75%, for example 5-65%, 20-50%, or more preferably 24-45%.

Preferably the granules contain total crystallinity, when equilibrated at 40-60% R^{H} in the range of 10-35%, for example, 15-35%, 18-30%, or more preferably 15-30%.

Preferably the granules contain a prbportion of double helix in the range 15-55%, for example 17-53%, 30-50%, 30-45%, or more preferably 25-50%.

Preferably the starch is from legume seed (e.g. pea, Pisum sativum; chickpea, Cicer arientinum; faba bean, Vicia faba, Phaseolus spp, lentil etc.). The examples below provide a wide range of novel and useful starch films from both wild-type and mutant pea (indicated by their gene symbols, where r and rug5 affect amylopectin; rb and rug4 affect substrate supply and lam affects amylose content) and from chickpea and faba bean. Some of the starches per se from pea mutants have previously been characterised at a molecular, genetic and biochemical level and by the effect of specific mutations on the supra-molecular structure of the starches (Bogracheva T. Y., Cairns P., Noel T. R., Hulleman S., Wang T. L., Morris V. J., Ring S. G. and Hedley C. L. (1999) Carb. Polym. 39, 303-314; Hedley C. L., Bogracheva T. Y. and Wang T. L. (2002) Starch/Starke, 54, 235-242; Hedley C. L., Bogracheva T. Y., Wang Y. and Wang T. L. (2001) Proceedings of Starch 2000: Structure and Function, Cambridge, pp 170-175; Bogracheva, T. et al “Structure-function relationships of pea starches associated with food and non-food uses”, 3^{rd} European Conference on Grain Legumes (1998) Valladolid); Redley, C. L. et al “A genetic approach to studying the morphology, structure and function of starch granules using pea as a model”, Starch Starke 54 (2002) 235-242; Bogracheva et al. “The granular structure of C-type pea starch and its role in gelatinisation”. Biopolymers, 45, 323-332, 1998; Bogracheva et al. “Structural studies of starches with different water contents”. Biopolymers, 64, 268-281, 2002. However none of these publications teach or suggest the structure function relationships, which form the basis of the present invention.

Table 1 herein shows the characteristics of various starches, including those which may be used in this aspect of the present invention. Unless otherwise stated, the plant designations refer to mutant peas.

Polyols

Polyols are polyhydric alcohols, ie. alcohols containing three or more hydroxyl groups and having the general formula CH₂OH(CHOH)ₙCH₂OH, where n may be from 2 to 5. Those having three hydroxyl groups (trihydric) are glycerols; those with more than three are called sugar alcohols. Commonly used polyols include sorbitol, maltitol and glycerol. One or more of these may be used in aspects of the invention, although the preferred plasticisers are liquid polyols at room temperature, such as glycerol (which has a m.p. of 17.8° C.).

Processing Variables

Those skilled in the art will be readily able to prepare starch films of the various aspects of the present invention in the light of the disclosure herein using specific methodology of their own choice. The results shown in the Examples below demonstrate certain preferred conditions (e.g. in the proportions of starch, glycerol—or other polyol—and water, and the heating, pressure, cooling, and drying conditions) for example to ensure an optimum amount of leached amylose.

Starch is provided, for instance, as described below wherein pea starches were ground and slurried at neutral or alkaline pH and centrifuged such as to separate a precipitate. This was then resuspended and sieved, before drying. Such techniques are well known to those skilled in the art and do not per se form part of the present invention.

It was found that film properties are highly dependent on the concentration of the starch suspension (Cₛₜ). Preferably the starch suspension in water and glycerol contains between about 0.1-6.0%, more preferably 0.4-5.5%, 1.0-5.2%, even more preferably between 2 and 4% starch.

Preferably the polyol concentration in the starch suspension is present at between 0 and 25%, for example 0-5%, 0.5-5.0% or 1.0-5.0%.

Note that all weights or ratios stated herein are dry weights or ratios, unless context demands otherwise.

The initial polyol/starch ratio may be between 0 and 2 but is more preferably 0.25 to 7, for example 0.25-1.5, or even more preferably 0.5 to 6, or 0.5 to 5, for example 0.5-1.5.

The heating step is known to be accompanied by the disruption of starch granular structure, granular swelling and partial or complete solubilisation of amylose. The extent to which each of these three events occurred was found to be dependent on the starch type and the method of heating, in particular on the final temperature. It was found that the heating temperature for film production should be at or higher than the structure disruption temperature, to allow swelling to be developed to a specific level and the optimal proportion of amylose to be leached from the granules.

Preferably the suspension is heated (or superheated) to between 80 and 180° C., for example 80 to 170° C., more preferably 85-150° C., more preferably to between 85-120° C., for example 80 to 100° C., gentle agitation being applied to the suspension during the heating. The rate of heating can be between 0.2-30° C./min, preferably 0.5-20° C./min and even more preferably 1.0-10° C. min. The maximum temperature can be maintained for 0-30 minutes.

Preferably following heating the starch paste may be centrifuged or exposed to a vacuum to remove air bubbles.

It is known that the drying-structuring step is accompanied by retrogradation, which is the formation of short- and long-order structures (double helices and crystallites). Retrogradation in the films of the present invention was found to be dependent, amongst other things, on the starch type and the heating, cooling, and storage conditions. Preferably the paste is cooled and dried at around 15-70° C., for example 15-20° C. or 20-60° C. As an option the temperature can be gradually reduced during drying from 40-60° C. to 15-25° C. The drying should be preferably on a flat surface. A drying time of at least 5 hours was generally used.

It was found that although films from different starches made with water and equilibrated at 44 or 58% RH were different, they were all relatively brittle. From the DSC and NMR studies it was found that the amorphous parts of these films were in a glassy state, which explains the brittle characteristics. Films equilibrated at 99% RH were partially in the rubbery state, which increases their flexibility.

Therefore, preferably the films made with pure water are conditioned by equilibrating at an RH of between 40 and 70% RR, preferably greater than 58%. Dry conditions should be avoided.

Films made with water plus glycerol and stored at 44-58% RH have a much higher proportion of plasticiser compared with films made with pure water and are, therefore, more flexible. The results of the present inventors show this high plasticiser content can be maintained stably over long periods.

It was also found that film properties were dependent on the amount of starch per cast area (S/CA) (which directly affects the final starch per film area (S/A)). Different CSt, S/CA, S/A and drying conditions resulted in films with different stabilities. A preferred S/CA range was 1 to 25 mg/cm², more preferably 4-14 mg/cm². In some embodiments the ratio may be 1-10 mg/cm², 2-9 mg/cm²; or 4-8 mg/cm².

A preferred S/A range was 1-45 mg/cm², for example 1-25 mg/cm², more preferably 4-15 mg/cm², for example 8-12 mg/cm².

Preferred Starches Films of the Invention

A further aspect of the invention relates to starch films obtained or obtainable by any of the processes and conditions described above.

Thus the invention provides a starch film obtained or obtainable by heating the preparation of starch from C-type starch granules as a suspension in water, water plus glycerol (or other polyols) to form a starch paste and cooling and drying the starch paste to form a starch film.

As discussed in more detail in the Examples below, preferred starch films of the invention formed generally from an amylose network which contained the granule remains as a filler. It is assumed that the cross-links of this network are crystallites and/or double helices (ordered structures) formed from the amylose molecules. The characteristics of the granule remains can be different, for example they are affected by their internal amylose content and the proportion of ordered structures.

Significant differences were found between films in the total proportion of ordered structures and in the arrangement of the crystallites, depending on the starch type and the processing conditions used. Usually films had B-type crystallinity (which is consistent with the results given by Myllarinen, P. et al “The crystallinity of amylose and amylopectin films”, Carbohydrate Polymers, vol 48, issue 1, (April 2002) 41-48; also Rindlav, A. et al “Formation of starch films with varying crystallinity”, Carbohydrate polymers 34 (1997) 25-30). It was found, however, that the type of polymorphs present in the films may be different.

Preferred films were “type 1” films as discussed in more detail in the examples below. Characteristic of such films is an enthalpy of melting (after 1 month storage) in the range of between 5-40, more preferably, 5-30, for example 10-30 J/g (film d.w.)/K under 58% RH which reflects are relatively high proportion of crystallinity. Preferred films of the present invention, such as those prepared with glycerol, have significantly higher 5.6 2-Theta peak then films made in pure water suggesting that the crystallites within such films are more elongated in one direction.

Preferred films according to the present invention comprise between at least 0-75% glycerol on a dry weight basis, for example 0-70%, or 0-40%. More preferably they contain between around 15-70% glycerol for example 20-65%, 20-35% or 15-35%. As discussed in the examples, the proportion of glycerol in most of the final films of the present invention was around 30% (g/g) as estimated using ‘method 1’ below. However using the more precise ‘method 2’ the figure was higher. Method 2 was the preferred method of determination.

Preferred films according to the present invention comprise between 10 and 95%, for example between 10 and 90%, for example 20-90% or 25-75% starch on a dry weight basis, and more preferably 10-70%, for example 10-60%.

The final polyol/starch ratio may be between 0 and 2 but is more preferably 0.1 to 7, for example 0.25-2, or even more preferably 0.5 to 6, for example 0.5-1.5.

Preferred films according to the present invention comprise between at least 5-55% water, more preferably between 10-52% water. However films may contain only 10-25% water.

The inventors have also demonstrated that films of the present invention may be provided which demonstrate high oxygen permeability, which may be advantageous in some applications, such as those discussed hereinafter (see also Forssell, P. et al “Oxygen Permeability of amylose and amylopectin films”, Carbohydrate Polymers, vol 47, issue 2, (February 2002) 125-129).

Preferred films according to the present invention consist, or consist essentially of starch, glycerol (or other polyols), and water i.e. do not contain other polymeric materials.

Preferred films according to the present invention have a % elongation of less than 60, more preferably less than 40, for example 2-30 or of 4-25.

Preferred films according to the present invention have a Yield stress of at least 0.01 MPA, for example between 1-100 MPA or 5-50 MPA. Most preferably the Yield stress is between 0.04 and 50 MPA, or 1 to 20 MPA.

Preferred films according to the present invention have a thickness of less than 1 mm, for example between 5-400, 5-200, or 30-180 m.

The films of the present invention may have highly water absorbant properties (see e.g. Table 12). As shown in the Examples below, the films prepared according to the present invention could be maintained substantially flat with a smooth texture and largely transparent. The films were also relatively stable when placed in water.

In addition to providing particular class of starch films having improved properties, the present inventors have realised that starch films having a relatively high plasticiser (e.g. water) content may be generally useful in medical applications—for example in wound dressings and the like. For example starch films can demonstrate flexibility, high plasticiser content, oxygen permeability, relatively low antigenicity, inherent bio-compatability, stability in water and also anti-bacterial properties.

Some of these applications are discussed below.

Wound Dressings

In another aspect the present invention provides use of a starch film as a wound dressing or the like, particularly a hydrated wound dressing. It further provides a wound dressing comprising, consisting of, or consisting essentially of, such films.

Preferred starch films for use in this aspect are those discussed above prepared from C-type starch granules and polyols. However other starch films, provided that they comprise between at least 5-55% water, more preferably between 10-52% water, may also be used in this aspect.

Such films are preferably obtained from starch suspensions heated (or superheated) to between 80 and 180° C., more preferably 80-170° C., 85-150° C., more preferably to 85-120° C., for example 85-100° C.

Preferred films according to the present invention comprise between at least 10-95%, for example 20-95%, starch on a dry weight basis and more preferably 25-75%. Preferred films for use in this aspect of the invention consist essentially of starch and water and optionally glycerol (or other polyol) i.e. do not contain other synthetic polymeric materials. They may, however, incorporate low concentrations of active ingredients, such as those described below.

Preferred films for use in this aspect of the present invention have a % elongation of less than 60, for example at least 2-45, 2-30, more preferably at least 4-25.

Preferred films may also have a Yield stress of at least 0.01 MPA, for example between 1-100 MPA or 5-50 MPA. Most preferably the Yield stress is between 0.04 and 50 MPA, or 1 to 20 MPA.

Where it is desired to further improve the mechanical strength of films, they may be employed as a coating on bandages or dressings of known type e.g. on the face of the bandage or dressing which contacts the skin. Thus, optionally, the starch film may be used or provided as a wound-dressing material or article comprising:

(i) a starch film, and

(ii) a support layer (for example a backing substrate) to which the film is optionally laminated.

The support layer may be integral with the film i.e. the film is prepared with the layer in situ.

The support layer may, for example, consist of a woven fabric, film, net, non-woven web the like. Backing substrates will be air-permeable. Possible backing or supporting substrates are disclosed, for example, in U.S. Pat. No. 5,613,942 (see also Table 13 herein).

The wound dressings of the present invention (such those including support layers) may include additional outer sheet materials, for example which are bacteria-impermeable.

The wound dressings of the invention may be perforated.

When used in the present aspects, films of the invention may incorporate antibodies or other active agents—for example antiseptic, antibiotic (antifungal or antibacterial) agents or anti-inflammatory agents, in addition to biocompatible colorants or stabilisers such as are well known in the art. Examples include silver metal and salts, and iodine. The hydrophilicity of the starch films facilitates the use of such agents and compounds.

The invention further provides such films and dressings for use in a method of treating or promoting healing of a wound, for example a burn, comprising contacting the wound with the film or dressing, or adhering the film or dressing to the skin of a patient in need of the same.

It will be appreciated that certain properties of starch films discussed herein may make them suitable for a variety of different dressing types and purposes, for example:
The maintenance of high humidity at the wound/dressing interface.
The removal of excess exudate.
Gaseous exchange.
Freedom from particles and toxic wound contaminants.
Good mechanical properties (strong, thin, flexible) for ease of application, maintenance and removal.
Pleasant skin-feel.
Antibacterial activity.
Non-toxic and low-allergenicity
Naturally produced—relatively inexpensive, sustainable

The starch films described herein can be used analogously to existing dressings or plasters for these purposes i.e. as dry dressings, moisture keeping dressings, and bioactive dressings. Bioactive dressings include antimicrobial dressings, interactive dressings, single-component biologic dressings, and combination products (see The Care of Wounds: A Guide for Nurses, Carol Dealey. Oxford, UK: Blackwell Science, Inc., 1994).

Preferably, starch films may be used to receive fluids diffusing to the dressing from the wound i.e. be used as an alternative to pastes, creams and ointments, nonpermeable or semipermeable membranes or foils, hydrocolloids, hydrogels, and combination products.

For example, films which absorb water can form a moist environment and so promote wound healing. These may be useful in the treatment of granulating or necrotic wounds, which have low to moderate exudate e.g. pressure sores, leg ulcers, surgical wounds, and minor burns. They may also facilitate rehydration and autolytic debridement of dry, sloughy or necrotic wounds. Thus the invention further provides such films and dressings for use in a method of promoting such wound hydration.

The invention further provides such films and dressings for use in a method of promoting wound drying e.g. for use in granulating wounds including leg ulcers, acute surgical wounds, sinuses and other cavity wounds (e.g. pressure sores) and removing exudate from “wet wounds”.

Other Microorganism-Controlling Films

As shown in Table 10 and Example 7, starch films as described herein can demonstrate potent antibiotic or antiseptic properties, causing substantial inhibition of bacterial growth. Thus the present invention further provides such starch films for use as medicaments e.g. is an antibiotic agents, and in particular antibacterial agents.

The invention particularly provides, in one aspect, use of a starch film for the control, growth inhibition, or destruction of a microorganism.

The invention further provides use of a starch film for the preparation of a medicament for the control, growth inhibition, or destruction of a microorganism e.g. for use in the treatment of a condition associated with that microorganism.

The films may be employed in any context where these properties may be required—for example in controlling microbial growth on surfaces to which the films are applied. In addition to wound dressings, the films may be useful for application sheets for topical drug delivery, sutures, disposable surgical appliances, and so on.

The invention will now be further described with reference to the following non-limiting Figure and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES & TABLES

FIG. 1 shows examples of three types of film under cross polarisers.

Table 1 herein shows the crystallinity and other characteristics of various starches used in the preparation of films of the present invention.

Table 2 shows the composition of various starch films of the invention before drying and after equilibration at 58% RH.

Table 3 shows various other properties of some of the starch films of the present invention, including appearance.

Table 4 shows the water uptake of starch films of the present invention.

Table 5 shows the properties of sorbitol and maltitol containing-films (equilibrated at 58% RH) prior to storage for 1 year (A) and after 1 year (B).

Table 6 shows the mechanical properties of a glycerol-containing film over 3 months.

Table 7 shows the effect of changing the glycerol concentration (glycerol/starch ratio) on the properties of various films. As can be seen favourable properties can be achieved up to quite high ratios of around 6 to 7. Max force, Yield stress and Elongation were determined at 3.0 mm/minute except for *=0.6 mm/min

Table 8 shows the effect of changing the weight of starch per area film (mg/cm²).

Table 9 shows the effect of drying temperature on film properties. The drying time at 60° C. was 24 hours. Table 10 shows the antibacterial properties of the films described herein.

Table 11 shows the effect of changing starch concentration (%) and weight per Film and per Cast Area (mg/cm²)

Table 12 shows the water absorbancy of films of the present invention.

Table 13 shows the characteristics of films made with inserted membranes. The majority of inserts tested, in different formats, gave excellent properties.

Table 14 shows the effect of inserting membranes into the films on mechanical properties and water absorption. All films were produced from 3.41% starch and 5.43% glycerol giving an initial starch/glycerol ratio of 1.59 with 8.1 mg starch/cm².

Table 15 shows the final composition of films containing glycerol, as measured using the preferred ‘method 2’ below.

Table 16 shows the final composition of films not containing polyol.

Table 17 shows the excellent Moisture Vapour Transmission Rate (MVTR) and absorbency properties of starch films containing glycerol, as compared with existing dressings.

A. The results are the mean of 5 films for each film type

B. Table B is reproduced from Thomas, S., and Loveless, P. (1997) ‘A comparative study of the properties of twelve hydrocolloid dressing’ World Wide Wound, Edition 1.0. Note the data is per 48 hours while data for A is per 24 hours.

### EXAMPLES

### Materials and Methods

Starches

Potato starch was obtained from Avebe Premier (UK). Low amylose (waxy) maize starch was obtained from Amioca (USA). The wheat starch was purchased from Sigma Chemical Co. (USA). These starches were used in comparative examples. Certain Pea starches were provided from Provital S. A. (Belgium).

Extraction and Purification of Pea Starches

All of the pea starches were extracted and purified as follows. The pea seeds were ground in a Cyclotec 1093 sample pin-mill (Sweden) and the resulting meal was slurried in water at a solid-liquid ratio of 10, the pH being adjusted to 8.2 with 0.1 M NaOH. The mixture was centrifuged at 2500 g (1500-3000 g) and the supernatant removed. The precipitate had a viscous green/yellow top layer (paste-like) and a solid main part. The viscous top layer was removed and the rest of the precipitate was re-suspended in distilled water. The centrifugation and separation procedure was repeated twice, until the viscous layer was no longer present. The resulting precipitate was then re-suspended in distilled water and screened sequentially through two sieves, 300 and 53μ. After screening through the 53μ sieve the slurry was left for 1-10 h, depending on the starch, at 4° C. The precipitate was collected, re-suspended in a large volume of water and vacuum filtered. The resulting starch cake was then air dried, during which any lumps were broken down progressively by hand.

For the starches from the r and rug5 mutants, the meal was air classified to separate the powder according to size and density before the starch extract was slurried with water. The air classification was carried out using an Alpine Multi-Plex® Laboratory Zigzag Classifier 100 MZR (Germany) with an air-flow of 46 m³/h, a motor speed of 7000 rpm and rate of sample feed of 0.7 kg/h.

Film Making

Starch suspensions in water or water/polyol mixtures were heated in a water bath or heating block with continuous gentle shaking to the required temperature (e.g. 98, 120, 150 or 160° C.) and kept at that temperature for 0-30 mins. The heating rate was 1-7° C./min. A standard water tap generated vacuum was applied to the starch pastes produced to remove air bubbles and then these were (˜2 min) poured into polystyrene petri dishes (10.3×10.3 cm) and the films dried on a flat surface at 20-70° C. The hot paste was poured into Petri dish such that it contained 1-25 mg starch/cm²

Starch Film Conditioning

Starch films were equilibrated in desiccators at 19-20° C. at different relative humidities (RH). To achieve the different humidity levels the film samples were stored in desiccators containing different saturated salt solutions: K₂CO₃—˜43% RH, NaBr—˜58-60% RH, K₂SO₄˜98-99% RH.

Determining Starch Film Structure and Properties

The structure and properties of the films were determined after conditioning. In addition, the structure and properties were also determined after starch films were dampened with tissues saturated in water; put into water; and put in salt solution (0.6M KCl).

For mechanical property testing, films were equilibrated at 20° C. at constant RH and cut into strips ˜70×7 mm. The thickness of the film strips was measured using a micrometer ˜every 1 cm along each strip.

The stress/strain measurements were performed using a Texture Analyser (TA. XT2). Stretching speed was originally 0.6 mm/min or (for data presented in Table 7 onwards) 3 mm/min. Yield stress and elongation were calculated as follows:

$$Yield Stress ( Mpa ) = \frac{Force at brake ( N )}{Area ( {mm}^{2} )} ,$$

where Area (mm²) strip width (mm)×thickness (mm)

$$Elongation ( \% ) = \frac{Extention ( at break )}{Length ( original )} \times 100$$

Proportion of double helices, crystalline type, total crystallinity, A/B crystallinity, and specific enthalpy of gelatinisation for starches (ΔH) were measured as described in Bogracheva et al, Biopolymers, 2002.

The crystalline type of the films was determined from wide range X-ray diffraction (XRD) measurements. A ARL X'TRA (ThermoARL) diffractometer with a fixed divergence slit was used. The experimental parameters were:—1.0/2.0 mm divergence slit, 0.5/0.2 mm receiving slit, X-ray tube—45 kV/44 mA. A microscope slide was used as a sample holder and the film strips were placed on the slide and run from 3.5 to 30 ° 2θ with 0.05 °2θ step sizes and 10 sec per step. The crystalline type was determined on the basis of peak profiles. The peak profiles were determined as described in Bogracheva et al, Biopolymers, 2002 (Ref 3 belowl.

Melting enthalpy of films was determined using DSC131 (Setaram). Films were powdered or cut in to small pieces with scissors in liquid nitrogen using a mortar and pestle and re-equilibrated at the desired % RH. 25-30 mg of the film powder was sealed in a stainless steel cuvette and equilibrated for 90 minutes at 7 or 12° C. Samples were then run to 180-220° C. at 5° C./min heating rate. An empty cuvette was used as a reference. The instrumental background was determined by running two empty cuvettes in the same conditions used for the samples. This background was subtracted and the area of the melting peak was calculated and used to calculate the specific enthalpy of melting (AH), using the same procedure as that used for starches.

All manipulations related to background subtractions, curve fitting, peak fitting and area calculations were performed using Origin software (Microcal, USA).

Determination of Moisture Vapour Transmission Rates and Absorption Properties using Paddington Cup Method

The tests were carried out by the Surgical Materials Testing Laboratory (SMTL) at Princess of Wales hospital in Bridgend Wales.

The Paddington Cup Method (modified Payne Cups) is a standard method for testing wound dressings, published in “Fluid Handling Properties of Wound Management Dressings” Surgical Materials Testing Lab. TM-65 (see also S. Thomas and P. Loveless. “A comparative study of the properties of twelve hydrocolloid dressings”. World Wide Wounds, July 1997.

The moisture vapour transmission test as described by SMTL is as follows: —

Five samples of each dressing of known weight are applied to Paddington cups to which are added 20 ml of a solution of sodium/calcium chloride containing 142 mmol/litre of sodium ions and 2.5 mmol/litre of calcium ions, here after known as solution A. The cups are weighed and placed in an incubator at 37±0.5° C. together with a tray containing 1 kg of freshly regenerated self indicating silica gel for a period of 24 hours. At the end of the test the cups are removed from the incubator, allowed to equilibrate to room temperature and reweighed. From these weighings the loss in weight due to the passage of moisture vapour through the dressing is determined. The base of each cup is then removed and any remaining fluid allowed to drain. The cup is then reweighed once again and the weight of the fluid retained by the dressing calculated by difference.

The tests were carried out with the film not in contact with the solution

Water Absorption Test

Approximately 14 mm×15 mm pieces of film were cut and weighed and measured. Excess water was added to cover the film and weighed. The jars were sealed and left to stand at room temperature for two hours. The films were removed and reweighed. The weight of water absorbed per unit area (g/cm²) and per unit weight of film (g/g) were calculated.

It was found on individual films that water absorption was not changed when films were left in water for ˜24 h.

In another test a film of 4% starch in 6% glycerol was heated to 120° C. in excess salt solution in a micro differential scanning calorimeter (DSC). The salt solution was used to simulate wound exudate. Films were immersed in the salt solution, heated, cooled and reheated over an 18 h period.

Determining Film Composition

Two methods were used.

For the results in Tables 2 and 3 the following method (‘method l’) was used for estimating the composition, with 3 replicates generally being carried out.

Water content: 5-10 mg of film were dried at 80° C. in vacuum oven to constant weight (˜1.5 to 3 days). The water content in the film was calculated by subtracting the weight of the dried film from the initial weight of the film and expressing the difference as a percentage of the initial film weight.

Starch and glycerol content: the proportion of starch plus glycerol in the film was calculated by subtracting the water content from 100%. The calculation for the proportion of starch and glycerol in the film was made assuming that the glycerol/starch ratio in the film remained the same as in the initial suspension.

For the results in Tables 5 onwards, the following more accurate method (‘method 2’) was used, with 3 replicates generally being carried out.

Water content: 5-10 mg of film was dried in a vacuum oven at 80-C for 20 hours. The percentage water content in the film was calculated by subtracting the weight of the dried film from the initial weight of the film and expressing the difference as a percentage of the initial film weight

Starch content: the amount of starch (on dry weight basis) in the paste poured into the cast and the total weight of film were measured. The concentration of starch in the film was calculated taking into account the weight of starch in the film and total film weight.

Glycerol content: the weight of glycerol was determined by subtracting the weight of water and starch in the film from the total weight of the film. This was used for calculating of the proportions of glycerol in the film.

For certain determinations (e.g. films coded 37 to 41 in Table 15) the water content was determined as above, and the starch and glycerol content was approximated by assuming the ratio of glycerol to starch in the films was equivalent to the ratio of glycerol to starch in the initial solution×0.9 (which figure was derived from the data provided elsewhere in the Table).

For films without glycerol, Methods 1 and 2 gave similar results. However it was found that for films with glycerol Method 2 was more accurate, as Method 1 tended to overestimate the final concentration of water, hence underestimate the final amounts of starch and glycerol. In particular the shorter drier times used for Method 2 (see S. H. D. Hulleman, F. H. P. Janssen, H. Feil. The role of water during plasticization of native starches. Polymer, Vol. 39, No. 10, pp. 2043-2048, 1998) meant that glycerol content was largely unaffected by the drying step. Additionally, the starch content was measured relatively accurately and independently from water or glycerol measurements.

### Preparation of Films with Inserts

Inserts of natural gauze, synthetic gauze, nylon netting (supplied by SMTL-Wales), sling bandage and miracloth (a material used for filtering made from rayon-polyester with an acrylic binding) were cut to fit into the 10.3 cm×10.3 cm Petri-dishes that were used as casts.

The inserts were incorporated in two ways.

i) The insert was placed into the Petri-dish and the starch/glycerol paste was poured on top.

ii) Starch/glycerol solution was poured into the Petri-dish and the insert was laid on top of the solution.

The films with inserts were dried and equilibrated in the usual way.

Antibacterial Testing of Films

The films tested were composed of an initial suspension of 3.4% starch 5.4% glycerol. The starch/cast area was 8 mg/cm², heating temp 98° C. The iodine was added as described in Example 5.

For the determination of antimicrobial efficacy, individual film surfaces were challenged with live Staphylococcus aureus inoculum and incubated for 24 hours according to a test method in accordance with the Japanese Film Sealing method JIS Z 2801. The inoculum was then washed off, appropriately serially diluted, plated up, incubated for 24 hours and then the viable count determined.

### Example 1

### Production of Starch Films

Optimisation of Properties

Extensive testing showed that films could be produced using the methods described herein having a wide variety of properties which could be tailored by varying the process parameters discussed above.

In respect of the final starch concentration in the films, a range within 1 to 6% was preferred. Very low values could produce only thin films, while very high values made for a viscous product which was difficult to cast (see e.g. Table 11).

In respect of the final glycerol concentration in the films, at least 1% was preferred. Above this level, mechanical properties were improved, although at higher levels (for example greater than around 20%) a more ‘jelly’ like film was produced (see e.g. Table 7).

In respect of the weight of starch/unit area in the films, the minimum amount was dictated by the requirement to cover the casting area, while at very high levels of starch it was found that the shrinkage could cause the film to split into fragments.

Structural Observations

It is believed that starch films prepared as described above are semi-crystalline structures and their network cross-links are formed by intermolecular crystallites, i.e. crystallites made with the involvement of different molecules. The amorphous areas are mainly responsible for the brittleness/elasticity of the films and these characteristics are strongly dependent on the glassy or rubbery state of the amorphous parts.

Table 1 shows the properties of the starches used in the present application, including amylose content and crystallinity.

It was deduced that three types of network can be formed during the production of cast starch films:—
1) An amylose network that incorporates the remains of swollen granules. This is common for normal starches that have a relatively high proportion of leached amylose.
2) A network formed by swollen granules. This is found when waxy (very low or no amylose) starches are used for the film.
3) A network produced when starch granules disappear during heating treatment or when they almost disappear, i.e. the granules swell to a very high degree.

In general, it was found that the first type of network was preferable for better mechanical properties of films. These properties, however, were highly dependent on the amount of leached and non-leached amylose and it was shown that an optimum amount of leached amylose is required for optimum mechanical properties of films. The results showed that the degree of amylose retrogradation was much higher than amylopectin retrogradation and that there is an optimum amount of retrogradation for optimal mechanical properties of films. The mechanical properties of films are also dependent on the characteristics of the granule remains.

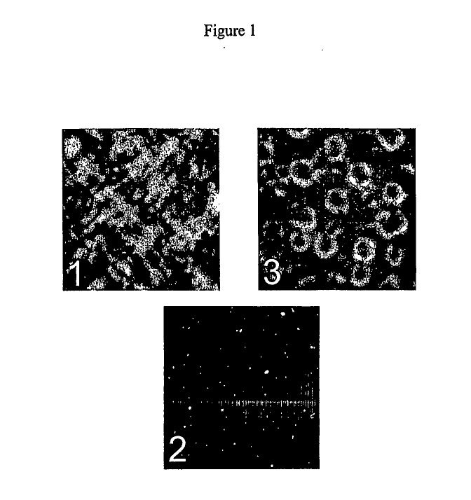

It was found that type-1 films had a relatively high proportion of short-ordered structures, about half of which were arranged in B-type crystallites. The amount of ordered structures in type-2 films was lower, a very small proportion being arranged in B-type crystallites. Type-3 films were found to have a similar proportion of short-ordered structures to type-1, but in this case the polymorph pattern was unusual. The arrangement of crystallites was also found to differ between the three types of film, which could be seen from the interference patterns obtained when the films were viewed under cross-polarisers in the light microscope. Typical images of all three types of film (FIG. 1) clearly show the difference in the crystallite arrangements.

The effect of glycerol on different types of film was found to be very different.

Table 2 shows the composition of various starch films tested in the present application.

When glycerol was added during the production of type-2 films, the resulting films shrank and generally had very poor properties. When glycerol was added during the production of type-1 films, good, intact films were usually produced that had good stability in water. When the ratio of glycerol/starch was 0.5 or more, the proportion of glycerol in the final film was always about 30% (g/g) as estimated with ‘method 1’ above, although increases in the glycerol/starch ratio resulted in film with progressively higher plasticiser contents as shown in the more accurate data obtained using ‘method 2’ above. In such films the addition of glycerol greatly decreased the film glass transition temperature, Young's modulus and yield stress, but significantly increased elongation. Once again the effects were dependent on the type of starch, with type C starches showing particularly advantageous properties.

### Example 2

### Stability in Water

If starch films are put into water at room temperature they can develop different characteristics, for example:

(i) losing integrity and developing a viscous-jelly appearance;

(ii) keeping integrity, but becoming very sticky, with parts of the films become glued to each other; or

(iii) keeping their integrity and not becoming sticky.

Table 3 summarises some of the properties of films of the present invention.

The type 1 films were shown to be consistently better than the other two types, and within this group glycerol could significantly improve the properties. The characteristics of films within this group, however, were dependent to a large extent on the type of starch, with those films being prepared from C-type starches giving the most consistently good properties.

In separate experiments, when films were heated in excess salt solution, the films did not break down on heating or reheating, but did absorb large amounts of water. This experiment showed the stability of the film on extended heating and reheating in excess water conditions.

### Example 3

### Biocompatible Properties

The starch/water films of the present invention showed good oxygen penetration, which was significantly increased by glycerol. Coupled with the properties described above, this makes them particularly suitable for medical applications, such as wound dressings.

As shown in Tables 4, 12, 14, and 17, starch films can be produced according to the methods described herein which are considerable water uptake—up to 70% of their weight. Such films can remove moisture from wet skin, after which the films dried to their original state. This can provide advantages, for example, in terms of keeping skin dry in wet wounds. Other films of the invention do not take up large amounts of moisture, and these may be preferred in other contexts.

Table 17 shows that films of the invention can also provide excellent moisture vapour transmission rates.

A further property observed with the films of the present invention, particularly those containing high amounts of plasticiser, was that they created a pleasing cooling sensation when applied to the skin.

### Example 4

### Storage of Sorbitol, Maltitol, and Glycerol Based Films

Starch films made using solutions of sorbitol and maltitol as plasticizers were stored in a desiccator maintaining a relative humidity of 58% at 20° C.

Observations regarding the appearance and properties of the films were made after a storage period of one year.

The results are shown in Table 5A and 5B.

After storage for 1 year, all films plasticized with sorbitol and maltitol were found to have undergone precipitation, the degree of which was proportionately higher when higher concentrations of these polyols were present in the films. It can therefore be concluded that levels of sorbitol and maltitol giving a polyol/starch ratio in excess of 0.46 are detrimental to the storage properties of these films.

By contrast films without or with glycerol (glycerol/starch 0.5-1.5) stored for in excess of 2 year at 58% RH did not change their appearance or textile properties. No precipitation was detected.

Additionally, as shown in Table 6, the mechanical properties of a glycerol containing film were not adversely affected by storage. These films were prepared at 98° C., dries at 20° C. Films, and equilibrated and maintained at 58% RH.

Other storage trials of the films (3.41% starch & 3.69% glycerol, 3.41% starch & 5.43% glycerol and 3.41% starch & 10.29% glycerol) showed that the film composition was substantially unchanged over the 80 day test.

### Example 5

### Preparation of Films with the Addition of Iodine

A starch/glycerol suspension was prepared and heated to 98° C. as previously described. 0.5 g of Alcoholic Iodine Solution B.P. (Iodine Tincture BP) was added to the paste, the paste was then shaken, de-aired and cast as described before. Six films were made. Natural Gauze was submerged to three of the films as previously described.

Iodine Tincture BP contained iodine BP 2.5% w/v, potassium iodine 2.5% w/v, ethanol (96%) BP 89% v/v and purified water BP to 100%. The initial starch suspension contained 3.41% starch, 5.8% glycerol, 1.7 Glycerol/Starch ratio. Starch per initial film area was 8.1 mg/cm². The final concentration of Iodine in films was assumed to be close to the concentration recommended for use in wound dressings prepared with iodine (1%). All films looked good, flat, flexible, relatively strong, they were a very dark purple colour and had a nice cool feeling when touched.

### Example 6

### Assessment of Strengthened Films

The results were as follows:

Gauzes: were very flexible and pliable. The inserts added strength although the films were still flexible. Both types of gauze (synthetic and natural) were easily wetted by the starch solution on pouring and readily incorporated into the solution.
Nylon inserts: were also very strong and pliable. Nylon wetted easily with the starch solution. Flexibility was apparently slightly lower than for the gauze films.
Bandage inserts: when produced as described in the methods these were relatively poor, with separation occurring between the bandage and film. However the use of other films or conditions may overcome these problems.
Miracloth inserts: very flexible and strong. The inserts added strength although the films were still flexible. The material was easily wetted by the starch solution on pouring and was readily incorporated into the film.

### Example 7

### Antibacterial Testing

As shown in Table 10, both types of starch film exhibited antibacterial properties.

The film without iodine reduced bacterial growth by log 1-1.5 units (i.e. 99.3%).

The film with iodine by log 3 units (equivalent to less than 99.9%) and so passed the JIS Z 2801 norm). The recorded count represent the detection error limit of the assay.

These results demonstrate that the films of the invention are not good media for bacterial growth, and indeed are strongly inhibitory of it. This demonstrates utility not only in wound dressings, but in anti-microbial products generally.

### REFERENCES

[1] Bogracheva T. Y., Cairns P., Noel T. R., Hulleman S., Wang T. L., Morris V. J., Ring S. G. and Hedley C. L. (1999) Carb. Polym. 39, 303-314.
[2] Bogracheva T. Y., Morris V. J., Ring S. G. and Hedley C. L. (1998) Biopol. 45, 323-332.
[3] Bogracheva T. Y., Wang Y. L., Wang T. L. and Hedley C. L. (2002) Biopol. 64, 268-281.
[4] Hedley C. L., Bogracheva T. Y. and Wang T. L. (2002) Starch/Starke, 54, 235-242.

[5] Hedley C. L., Bogracheva T. Y., Wang Y. and Wang T. L. (2001) Proceedings of Starch 2000: Structure and Function, Cambridge, pp 170-175.

TABLE 1
Starches-characteristics

| | | | | | Proportion A/B | | | | | |
|---|---|---|---|---|---|---|---|---|---|---|
| | | | Crystallinity | | crystallinity in | | | | | |
| | | | of | | starches | | | | | |
| Plant | | | starches | | equilibrated in dry | | | | | |
| (mutant) | | | equilibrated | Crystal | and wet conditions | | Tp, C. | Tc, C. | A-L/Tp | A-L/Tc |
| type and | | Double | at | line | | Excess | Excess | Excess | Excess | Excess |
| line | Amylose % | Helix % | 43% RH | type | 43% RH | water | H₂O | H₂O | H₂O | H₂O |
| Method | Megazine | NMR, | XRD | XRD | XRD | DSC | DSC | DSC | | |
| | | DSC | | DSC | | | | | | |
| WT pea | 30 | 40 | 23 | C | 53/47 | 42/58 | 62 | 77 | — | — |
| rug4-b | 30 | 41 | 24 | C | 53/47 | 37/63 | 63 | 75 | — | — |
| rb | 20 | 48 | 27 | C | 57/43 | 49/51 | 66 | 77 | — | — |
| lam-c | 10 | 49 | 27 | C | 30/70 | 26/73 | 59 | 68 | — | — |
| | | | | | | | 64 | 73 | | |
| rug5-a | 39 | 32 | 18 | C | 47/53 | n/d | 49 | 110 | — | — |
| f-57 | 57 | 22 | 20 | C | 90/10 | n/d | 72 | 100 | — | — |
| Faba | 32 | 35 | 29 | C | n/d | 56/44 | 64 | 80 | — | — |
| beans | | | | | | | | | | |
| Chick | 25 | 39 | 24 | C | n/d | 60/40 | 62 | 79 | — | — |
| peas | | | | | | | | | | |
| Potato | 21 | 48 | 24 | B | 0/100 | 0/100 | 62 | 72 | — | — |
| Wheat | 27 | 32 | 21 | A | 100/0 | 100/0 | 58 | 70 | 95 | 101 |
| ‘waxy’ | 1-2 | 48 | 32 | A | 100/0 | 100/0 | 71 | 78 | — | — |
| maize | | | | | | | | | | |

WT = Wild type
A = A type starch
B = B type starch
A-L = amylose-lipid complex
Tp = Peak gelatinisation temperature
Tc = Completion of gelatinisation temperature

TABLE 2
Comparison of composition of films before drying and after equilibration
Drying at 20° C. unless stated

| | | | | | | mg | ΔH | | |
|---|---|---|---|---|---|---|---|---|---|
| | | % | % | % | | St/ | J/gK | | |
| | | Starch | Water | Glycerol | | cm² | g/ | | |
| | Starch/Plasticiser/ | (dwt | (dwt | (dwt | Gl/St | of | d.w.of | | |
| Run | heating temp. | basis) | basis) | basis) | Ratio | film | film | DH % | RH, % |
| | rug4 | | | | | | | | |
| Fr124 | 3.0rug4/H2O/98 | | | | | | | | |
| | (18/9/02-6) | | | | | | | | |
| | initial | 2.69 | 97.31 | 0 | 0 | | | | |
| | film | 85.34 | 14.66 | 0 | | 6.6 | 13.9 | 32 | 58 |
| Fr129 | 0.5rug4/H2O/98 (19/9/02- | | | | | | | | |
| | 1) | | | | | | | | |
| | initial | 0.47 | 99.53 | 0 | 0 | | | | |
| | film | 90.40 | 9.60 | 0 | | 1.1 | 13.1 | 31 | 58 |
| Fr179 | 2.1rug4/H2O/120° (10/9/02- | | | | | | | | |
| | 2) | | | | | | | | |
| | initial | 1.90 | 98.10 | 0 | 0 | | | | |
| | film | 87.38 | 12.62 | 0 | | 4.2 | 14.8 | 34 | 58 |
| Fr164 | 2.1rug4/H2O/160° | | | | | | | | |
| | (17/12/02-2) | | | | | | | | |
| | initial | 1.86 | 98.14 | 0 | 0 | | | | |
| | film | 86.83 | 13.17 | 0 | | 4.0 | 15.5 | 36 | 58 |
| Fr168 | 2.1rug4/0.5% G/98 | | | | | | | | |
| | (26/7/02-2) | | | | | | | | |
| | initial | 1.92 | 97.59 | 0.49 | 0.26 | | | | |
| | film | 71.07 | 10.79 | 18.14 | | 3.9 | N/d | N/d | 58 |
| Fr132 | 2.1rug4/1% G/98 | | | | | | | | |
| | (11/6/02-6) | | | | | | | | |
| | initial | 1.88 | 97.01 | 1.11 | 0.51 | | | | |
| | film | 55.84 | 11.19 | 32.97 | | 3.8 | 20.7 | N/d | 58 |
| Fr119 | 2.1rug4/1% G/98 | | | | | | | | |
| | (26/6/02-1) | | | | | | | | |
| | initial | 1.91 | 97.11 | 0.98 | 0.59 | | | | |
| | film | 57.60 | 12.85 | 29.55 | | 3.8 | 27.4 | N/d | 58 |
| Fr166 | 2.1rug4/1% G/160° | | | | | | | | |
| | (18/12/02-3) | | | | | | | | |
| | initial | 1.86 | 97.16 | 0.98 | 0.53 | | | | |
| | film | 46.57 | 28.90 | 24.53 | | 4.1 | N/d | N/d | 58 |
| Fr120 | 2.1rug4/2% G/98 | | | | | | | | |
| | (26/7/02-4) | | | | | | | | |
| | initial | 1.91 | 96.13 | 1.96 | 1.03 | | | | |
| | film | 32.60 | 33.94 | 33.46 | | 3.9 | 22.5 | N/d | 58 |
| Fr122 | 2.1rug4/3% G/98 | | | | | | | | |
| | (26/7/02-5) | | | | | | | | |
| | initial | 1.94 | 95.13 | 2.93 | 1.51 | | | | |
| | film | 21.04 | 47.18 | 31.78 | | 4.0 | 20.0 | N/d | 58 |
| Fr035 | 2.1rug4/H2O/98 (11/6/02- | | | | | | | | |
| | 2) | | | | | | | | |
| | initial | 1.87 | 98.13 | 0 | 0 | | | | |
| | film | 78.30 | 21.70 | 0 | 0 | 3.8 | 15.7 | 36.5 | 99 |
| Fr036 | 2.1rug4/H2O/98 (11/6/02- | | | | | | | | |
| | 2) | | | | | | | | |
| | initial | 1.87 | 98.13 | 0 | 0 | | | | |
| | film | 72.10 | 27.90 | 0 | 0 | 3.8 | 23.2 | 54 | 99 |
| | lam | | | | | | | | |
| Flm100 | 1.5lam/H2O/98 | | | | | | | | |
| | (2/8/02-2) | | | | | | | | |
| | initial | 1.30 | 98.70 | 0 | 0 | | | | |
| | film | 87.25 | 12.75 | 0 | | 2.5 | 5.8 | 14 | 58 |
| Flm103 | 2.1lam/H2O/98 (30/7/02- | | | | | | | | |
| | 2) | | | | | | | | |
| | initial | 1.84 | 98.16 | 0 | 0 | | | | |
| | film | 86.72 | 13.28 | 0 | | 3.2 | N/d | N/d | 58 |
| Flm95 | 2.1lam/H2O/98 (16/8/02- | | | | | | | | |
| | 1) | | | | | | | | |
| | initial | 1.84 | 98.16 | 0 | 0 | | | | |
| | film | 86.41 | 13.59 | 0 | | 3.2 | 9.8 | 23 | 58 |
| Fl180 | 2.0lam/H2O/120 (12/9/02- | | | | | | | | |
| | 1) | | | | | | | | |
| | initial | 1.78 | 98.22 | 0 | 0 | | | | |
| | film | 88.61 | 11.39 | 0 | | 4.1 | 8.8 | 20.5 | 58 |
| Fl140 | 2.1lam/1% G/98 (2/8/02- | | | | | | | | |
| | 6) | | | | | | | | |
| | initial | 1.87 | 97.18 | 0.95 | 0.51 | | | | |
| | film | 56.08 | 15.43 | 28.49 | | 3.6 | 12.2 | N/d | 58 |
| Fl141 | 2.1lam/1% G/98 | | | | | | | | |
| | (16/8/02-4) | | | | | | | | |
| | initial | 1.84 | 97.18 | 0.98 | 0.53 | | | | |
| | film | 55.33 | 15.20 | 29.47 | | 3.6 | 20.6 | N/d | 58 |
| Flm97 | 2.1lam/2% G/98 | | | | | | | | |
| | (16/8/02-5) | | | | | | | | |
| | initial | 1.84 | 96.20 | 1.96 | 1.07 | | | | |
| | film | 25.62 | 47.09 | 27.29 | | 3.9 | 21 | N/d | 58 |
| Fl037 | 2.0/lam/H20/98 | | | | | | | | |
| | initial | 1.78 | 98.22 | 0 | 0 | | | | |
| | film | 79.3 | 20.7 | 0 | 0 | 3.4 | 7.0 | 16 | 99 |
| | Wild type | | | | | | | | |
| Fw162 | 2.1w/t/H2O/98° (1/10/02- | | | | | | | | |
| | 2) | | | | | | | | |
| | initial | 1.79 | 98.21 | 0 | 0 | | | | |
| | film | 86.60 | 13.40 | 0 | 0 | 3.9 | 11.6 | 27 | 58 |
| Fw172 | 2.1w/t/H2O/98° (1/10/02- | | | | | | | | |
| | 1) | | | | | | | | |
| | initial | 1.79 | 98.21 | 0 | 0 | | | | |
| | film | 85.60 | 14.40 | 0 | 0 | 3.9 | 14.1 | 33.5 | 58 |
| Fw001 | 2.1w/t/1% G/98° (1/10/02- | | | | | | | | |
| | 6) | | | | | | | | |
| | initial | 1.79 | 97.23 | 0.98 | 0.55 | | | | |
| | film | 56.63 | 12.37 | 31.00 | | 4.0 | 16.8 | N/d | 58 |
| Fw034 | 2.1w/t/H2O/98° (1/10/02- | | | | | | | | |
| | 2) | | | | | | | | |
| | initial | 1.79 | 98.21 | 0 | 0 | | | | |
| | film | 78.60 | 21.40 | 0 | | 3.9 | 8.2 | 19 | 99 |
| — | 2.1w/t/2% G/98° (11/4/03- | | | | | | | | |
| | 1) | | | | | | | | |
| | initial | 1.79 | 96.25 | 1.96 | 1.10 | | | | |
| | film | 35.53 | 25.56 | 38.91 | | 3.9 | N/d | N/d | 58 |
| — | 2.1 Rw/t/1% G/98° | | | | | | | | |
| | (7/5/03-6) | | | | | | | | |
| | Initial-(st corr-95%) | 1.74 | 98.26 | 0.98 | 0.60 | | | | |
| | Film-(st corr-95%) | 48.7 | 21.38 | 27.32 | | 3.9 | N/d | N/d | 58 |
| | WT/H20/160 | | | | | | | | |
| | 15/3/04-1,2,3 | | | | | | | | |
| | Initial | 1.77 | 98.23 | 0 | 0 | | | | 58 |
| | film | 88.43 | 11.57 | 0 | | 3.8 | | | |
| | WT/1% G/166 | | | | | | | | |
| | 18/03/04-1,2,3 | | | | | | | | |
| | Initial | 1.77 | 97.25 | 0.98 | 0.55 | | | | 58 |
| | film | 56.41 | 12.36 | 31.23 | | 3.8 | | | |
| | WT/H20/166 | | | | | | | | |
| | 24/3/04-1,2,3 | | | | | | | | |
| | Initial | 1.77 | 98.23 | 0 | 0 | | | | 58 |
| | film | 88.45 | 11.55 | 0 | | 3.8 | | | |
| | WT/1% G/98 | | | | | | | | |
| | 16/04/04-1 | | | | | | | | |
| | Initial | 2.11 | 96.91 | 0.98 | 0.46 | | | | 58 |
| | film | 57.69 | 15.52 | 26.79 | | 4.6 | | | |
| | WT/1% G/98 | | | | | | | | |
| | 16/04/04-2 | | | | | | | | |
| | Initial | 2.11 | 96.92 | 0.97 | 0.46 | | | | 58 |
| | film | 57.88 | 15.52 | 26.61 | | 4.6 | | | |
| | WT/1% G/98 | | | | | | | | |
| | 16/04/04-3,4 | | | | | | | | |
| | Initial | 2.53 | 96.50 | 0.97 | 0.38 | | | | 58 |
| | film | 64.19 | 11.20 | 24.61 | | 5.5 | | | |
| | WT/1% G/98 | | | | | | | | |
| | 16/04/04-5,6 | | | | | | | | |
| | Initial | 3.37 | 95.67 | 0.96 | 0.29 | | | | |
| | film | 69.52 | 10.67 | 19.80 | | 7.3 | | | 58 |
| | WT/H20/98 | | | | | | | | |
| | 19/04/04-1 | | | | | | | | |
| | Initial | 5.05 | 94.95 | 0 | 0 | | | | |
| | film | 87.57 | 12.43 | 0 | | 9.6 | | | 58 |
| | WT/H20/98 | | | | | | | | |
| | 19/04/04-2 | | | | | | | | |
| | Initial | 5.05 | 94.95 | 0 | 0 | | | | |
| | film | 87.57 | 12.43 | 0 | | 7.2 | | | 58 |
| | WT/H20/98 | | | | | | | | |
| | 19/04/04-3,4 | | | | | | | | |
| | Initial | 3.37 | 96.63 | 0 | 0 | | | | |
| | film | 87.18 | 12.82 | 0 | | 7.3 | | | 58 |
| | WT/H20/98 | | | | | | | | |
| | 19/04/04-5,6 | | | | | | | | |
| | Initial | 4.21 | 95.79 | 0 | 0 | | | | |
| | film | 86.92 | 13.08 | 0 | | 8.9 | | | 58 |
| | WT/3% G/98 | | | | | | | | |
| | 20/04/04B-1,2 | | | | | | | | |
| | Initial | 2.53 | 94.56 | 2.91 | 1.16 | | | | |
| | film | 22.97 | 50.58 | 26.42 | | 4.3 | | | 58 |
| | WT/4% G/98 | | | | | | | | |
| | 20/04/04B-3,4 | | | | | | | | |
| | Initial | 3.37 | 92.79 | 3.84 | 1.14 | | | | |
| | film | 25.70 | 45.02 | 29.28 | | 5.8 | | | 58 |
| | WT/5% G/98 | | | | | | | | |
| | 20/04/04B-5,6 | | | | | | | | |
| | Initial | 4.21 | 91.04 | 4.75 | 1.13 | | | | |
| | film | 26.34 | 43.94 | 29.72 | | 7.4 | | | 58 |
| | WT/1% sorbitol/98 | | | | | | | | |
| | 21/04/04-1 | | | | | | | | |
| | Initial | 1.77 | 97.25 | 0.98 | 0.55 | | | | |
| | film | 58.28 | 9.45 | 32.27 | | 3.84 | | | 58 |
| | WT/2% sorbitol/98 | | | | | | | | |
| | 21/04/04-3 | | | | | | | | |
| | Initial | 1.77 | 96.27 | 1.96 | 1.11 | | | | |
| | film | 42.58 | 10.27 | 47.16 | | 3.84 | | | 58 |
| | WT/3% sorbitol/98 | | | | | | | | |
| | 21/04/04-6 | | | | | | | | |
| | Initial | 1.77 | 95.29 | 2.94 | 1.66 | | | | |
| | film | 33.38 | 11.17 | 55.45 | | 3.82 | | | 58 |
| | WT/1% maltitol/98 | | | | | | | | |
| | 28/04/04-1 | | | | | | | | |
| | Initial | 1.77 | 97.25 | 0.98 | 0.55 | | | | |
| | film | 58.34 | 9.36 | 32.30 | | 3.84 | | | 58 |
| | WT/1% maltitol/98 | | | | | | | | |
| | 28/04/04-3 | | | | | | | | |
| | Initial | 1.77 | 96.27 | 1.96 | 1.11 | | | | |
| | film | 42.57 | 10.31 | 47.14 | | 3.84 | | | 58 |
| | Pea (Provital) | | | | | | | | |
| | WTPea/H20/98 | | | | | | | | |
| | initial | 1.88 | 98.12 | 0 | 0 | | | | |
| | film | | | | | 4.1 | | | 58 |
| | WTPea1% G/98 | | | | | | | | |
| | initial | 1.88 | 97.14 | 0.98 | 0.52 | | | | |
| | film | | | | | 4.1 | | | 58 |
| | r57 | | | | | | | | |
| Fr158 | 2.1r 57/H2O | | | | | | | | |
| | (11/12/02-1) | | | | | | | | |
| | initial | 1.85 | 98.15 | 0 | 0 | | | | |
| | film | 86.35 | 13.65 | 0 | | 4.0 | 5.0 | 12 | 58 |
| Fr183 | 2.1r 57/1% G/160° | | | | | | | | |
| | (11/12/02-3) | | | | | | | | |
| | initial | 1.86 | 97.16 | 0.98 | 0.53 | | | | |
| | film | 56.13 | 14.29 | 29.58 | | 4.1 | 5.9 | N/d | 58 |
| | Rug5 | | | | | | | | |
| F5135 | 2.1rug5/H2O/120° | | | | | | | | |
| | (3/10/02-4) | | | | | | | | |
| | initial | 1.92 | 98.08 | 0 | 0 | | | | |
| | film | 85.71 | 14.29 | 0 | | 4.2 | 11.3 | 26 | 58 |
| F5160 | 2.1rug5/H2O/160° | | | | | | | | |
| | (27/11/02-2) | | | | | | | | |
| | initial | 1.87 | 98.13 | 0 | 0 | | | | |
| | film | 86.29 | 13.71 | 0 | | 4.3 | 13.7 | 32 | 58 |
| F5184 | 2.1rug5/1% G/120° | | | | | | | | |
| | (3/10/02-5) | | | | | | | | |
| | initial | 1.90 | 97.12 | 0.98 | 0.52 | | | | |
| | film | 57.38 | 23.02 | 29.60 | | 4.2 | N/d | N/d | 58 |
| F5185 | 2.1rug5/1% G/160° | | | | | | | | |
| | (5/12/02-1) | | | | | | | | |
| | initial | 1.85 | 97.17 | 0.98 | 0.53 | | | | |
| | film | 55.53 | 15.05 | 29.42 | | 4.1 | 19.6 | N/d | 58 |
| | Wheat | | | | | | | | |
| Fw126 | 2.1wheat/H2O/120 | | | | | | | | |
| | (4/9/02-1) | | | | | | | | |
| | initial | 1.79 | 98.21 | 0 | 0 | | | | |
| | film | 87.33 | 12.67 | 0 | | 3.9 | 23.5 | 55 | 58 |
| Fw130 | 2.1wheat/H2O/120 | | | | | | | | |
| | (9/10/02-3) | | | | | | | | |
| | initial | 1.88 | 98.12 | 0 | 0 | | | | |
| | film | 87.25 | 12.75 | 0 | | 4.1 | 27.8 | 65 | 58 |
| Fw186 | 2.1wheat/1% G/120 | | | | | | | | |
| | (9/10/02-6) | | | | | | | | |
| | initial | 1.88 | 97.14 | 0.98 | 0.52 | | | | |
| | film | 57.94 | 11.86 | 30.20 | | 4.1 | 10.4 | N/d | 58 |
| Fw008 | 2.1wheat/H2O/160 | | | | | | | | |
| | (20/11/02-1) | | | | | | | | |
| | initial | 1.90 | 98.10 | 0 | 0 | | | | |
| | film | 88.59 | 11.41 | 0 | | 4.1 | | | 58 |
| | Wheat/H2O/166 | | | | | | | | |
| | 25/03/04-1 | | | | | | | | |
| | Initial | 1.88 | 98.12 | 0 | 0 | | | | |
| | film | 89.48 | 10.52 | 0 | | 4.0 | | | 58 |
| | Wheat/1% G/166 | | | | | | | | |
| | 26/3/04-3 | | | | | | | | |
| | Initial | 1.88 | 97.14 | 0.98 | 0.52 | | | | |
| | film | 54.97 | 16.37 | 28.66 | | 4.1 | | | 58 |
| | Waxy maize | | | | | | | | |
| Fm170 | 2.1WM/H2O/98 | | | | | | | | |
| | (27/9/02-2) | | | | | | | | |
| | initial | 1.82 | 98.18 | 0 | 0 | | | | |
| | film | 88.17 | 11.83 | 0 | | 4.0 | 6.1 | 14 | 58 |
| Fm038 | 2.1WM/H2O/98 | | | | | | | | |
| | (27/9/02-2) | | | | | | | | |
| | initial | 1.82 | 98.18 | 0 | 0 | | | | |
| | film | 77.36 | 22.64 | 0 | | 4.0 | 4.8 | 11 | 99 |
| Fm120 | WM/1% G/98 | | | | | | | | |
| | 27/9/02-6 | | | | | | | | |
| | initial | 1.83 | 97.19 | 0.98 | 0.54 | | | | |
| | film | 47.14 | 27.62 | 25.24 | | 4.0 | 15.7 | N/d | 58 |
| | Potato | | | | | | | | |
| Fp139 | 1.8potato/H2O/98 | | | | | | | | |
| | (6/8/02-3) | | | | | | | | |
| | initial | 1.53 | 98.47 | 0 | 0 | | | | |
| | film | 88.64 | 11.36 | 0 | | 2.6 | 13.5 | 31 | 58 |
| Fpt102 | 1.5potato/1% G/98 | | | | | | | | |
| | (6/8/02-2) | | | | | | | | |
| | initial | 1.27 | 97.73 | 0.99 | 0.78 | | | | |
| | film | 36.00 | 35.94 | 28.06 | | 2.7 | 18.2 | N/d | 58 |
| | Rb (pea mutant) | | | | | | | | |
| Fb002 | 2.1% rb/H2O/98 | | | | | | | | |
| | (12/3/03-4) | | | | | | | | |
| | initial | 1.76 | 98.24 | — | 0 | | | | |
| | film | 86.57 | 13.43 | — | | 3.8 | 14.5 | 34 | 58 |
| Fb005 | 2.1% rb/H2O/150 | | | | | | | | |
| | (27/3/03-1) | | | | | | | | |
| | initial | 1.78 | 98.22 | — | 0 | | | | |
| | film | 87.31 | 12.69 | — | | 3.9 | 14.4 | 33.5 | 58 |
| Fb004 | 2.1% rb/1% G/98 | | | | | | | | |
| | (12/3/03-6) | | | | | | | | |
| | initial | 1.76 | 97.26 | 0.98 | 0.56 | | | | |
| | film | 53.56 | 16.61 | 29.83 | | 3.8 | 17.3 | N/d | 58 |
| | Faba bean | | | | | | | | |
| | Faba bean/H20/98 | | | | | | | | |
| | 14/4/04A-4,5,6 | | | | | | | | |
| | initial | 1.88 | 98.12 | 0 | 0 | | | | |
| | film | 87.93 | 12.07 | 0 | | 4.1 | | | 58 |
| | Faba bean/1% G/98 | | | | | | | | |
| | 14/4/04B-4,5,6 | | | | | | | | |
| | initial | 1.88 | 96.95 | 1.17 | 0.63 | | | | |
| | film | 44.22 | 28.26 | 27.52 | | 4.1 | | | 58 |
| | Chickpea (desi | | | | | | | | |
| | Heera) | | | | | | | | |
| | Chickpea/H20/98 | | | | | | | | |
| | 14/4/04A-1,2,3 | | | | | | | | |
| | initial | 1.89 | 98.11 | 0 | 0 | | | | |
| | film | 87.79 | 12.21 | 0 | | 4.1 | | | 58 |
| | Chickpea/1% G/98 | | | | | | | | |
| | 14/4/04B-1,2,3 | | | | | | | | |
| | initial | 1.89 | 96.94 | 1.17 | 0.62 | | | | |
| | film | 49.57 | 19.73 | 30.69 | | 4.1 | | | 58 |

| | | | | | | | mg | ΔH | |
|---|---|---|---|---|---|---|---|---|---|
| | | | % | % | % | | ST// | J/gK | |
| | | | Starch | Water | Glycerol | | cm² | g- | |
| | Starch/Plasticiser/heating | Drying | (dwt | (dwt | (dwt | | of | d.w.of | |
| Run | temp. | T, C. | basis) | basis) | basis) | Gl/St | film | film | RH, % |
| | WT pea - drying | | | | | | | | |
| | temps 40° C. & 60° C. | | | | | | | | |
| | WT/H20/98 | | | | | | | | |
| | 15/04/04-B 1,2,3 | | | | | | | | |
| | initial | 40 | 1.77 | 98.23 | 0 | 0 | | | |
| | film | | 88.77 | 11.23 | 0 | | 3.8 | | 58 |
| | WT/1% G/98 | | | | | | | | |
| | 15/04/04-A1,2,3 | | | | | | | | |
| | initial | 40 | 1.77 | 97.25 | 0.98 | 0.55 | | | |
| | film | | 43.44 | 32.51 | 24.05 | | 3.8 | | 58 |
| | WT/1% G/98 | | | | | | | | |
| | 20/04/0-A 4,5,6 | | | | | | | | |
| | initial | 60 | 1.77 | 97.25 | 0.98 | 0.55 | | | |
| | film | | 51.35 | 20.22 | 28.43 | | 3.9 | | 58 |
| | WT/H20/98 | | | | | | | | |
| | 20/04/0-A 1,2,3 | | | | | | | | |
| | initial | 60 | 1.77 | 98.23 | 0 | 0 | | | |
| | film | | 89.21 | 10.79 | 0 | | 3.8 | | 58 |

TABLE 3
Starch films, 58% RH

| | | | | Max | | | | | | |
|---|---|---|---|---|---|---|---|---|---|---|
| | Av.film | | | Heat | | Film size | mg St/ | | | |
| | Thickness | Starch | | Temp | Gl/st | (original = 10.3 cm × | cm² | | ‘In water’ | |
| Starch | um (+/−5) | Conc % | Plasticizer | C. | g/g | × 10.3 cm) | film | Appearance | stability | DH % |
| rug4/Fr129 | | 0.47 | H2O | 98 | — | 10.3 × 1-.3 | 0.99 | Transp, | | |
| | | | | | | | | flat | | |
| rug4 | | 0.89 | H2O | 98 | — | 10.2 × 10.2 | 2.02 | Transp, | | |
| | | | | | | | | flat | | |
| rug4 | | 1.33 | H2O | 98 | — | 10.2 × 10.2 | 3.01 | Transp, | | |
| | | | | | | | | flat | | |
| rug4 | 41 | 1.78 | H2O | 98 | — | | 4.03 | | | |
| rug4 | | 1.86 | H2O | 98 | — | 10.3× | 4.20 | Transp, | Good | |
| | | | | | | | | flat | | |
| rug4/Fr179 | 32 | 1.90 | H2O | 120 | — | | 4.16 | | | |
| rug4 | 30 | 1.86 | H2O | 160 | — | 9.9× | 4.04 | Milky, | Bad | |
| | | | | | | | | flat | | |
| rug4/Fr168 | | 1.92 | 0.5% G | 98 | 0.26 | 9.5× | 3.89 | Transp, | Very good | |
| | | | | | | | | flat | | |
| rug4/Fr132 | 50 | 1.90 | 1% G | 98 | 0.51-0.59 | 9.2 × 9.3 | 3.84 | Transp, | Very good | |
| or Fr119 | | | | | | | | flat | | |
| rug4/Fr166 | | 1.86 | 1% G | 160 | 0.53 | 9 × 9 | 4.07 | Milky, | Bad | |
| | | | | | | | | flat | | |
| rug4/Fr120 | 71 | 1.91 | 2% G | 98 | 1.03 | 8.8 × 8.9 | 3.88 | Transp, slightly | Very good | |
| | | | | | | 8.5 × 8.5 | | wrinkled | | |
| rug4/Fr122 | 100 | 1.94 | 3% G | 98 | 1.51 | 7.3 × 7.5 | 3.95 | Transp, slightly | Very good | |
| | | | | | | | | wrinkled | | |
| rug4 | | 2.22 | H2O | 98 | — | 10.1 × 10.1 | 5.11 | Transp, | Good | |
| | | | | | | | | flat | | |
| rug4 | | 2.69 | H2O | 98 | — | 9.8 × 9.8 | 6.60 | Transp, | Good | |
| | | | | | | | | flat | | |
| Lam/Flm100 | | 1.30 | H2O | 98 | — | | 2.51 | | | |
| Lam | 25 | 1.60 | H2O | 98 | — | 10.2 × 10.3 | 3.47 | Transp, slightly | Very | |
| | | | | | | | | wrinkled | bad | |
| Lam/Flo37 | | 1.78 | H2O | 98 | — | | 3.44 | Transp, | Very | |
| | | | | | | | | plain | bad | |
| Lam/Flm103 | 23 | 1.84 | H2O | 98 | — | 10.2 × 10.2 | 3.19 | Transp, slightly | Very | |
| or Flm95 | | | | | | | | wrinided | bad | |
| Lam/Fl180 | | 1.78 | H2O | 120 | — | | 4.05 | | | |
| Lam/Fl140 | 42 | 1.85 | 1% G | 98 | 0.51-0.53 | 8.8 × 9 | 3.56 | Transp, slightly | Very | |
| or Fl141 | | | | | 0.53 | | | wrinkled | bad | |
| Lam/Flm97 | | 1.84 | 2% G | 98 | 1.07 | | 3.92 | | | |
| Wt pea/Fw162 | 37 | 1.79 | H2O | 98 | — | 10.1 × 10.1 | 3.90 | Transp., flat | Good | |
| or Fw172 | | | | | | | | | | |
| or Fw034 | | | | | | | | | | |
| Wt pea/Fw001 | 54 | 1.79 | 1% G | 98 | 0.55 | 9 × 9 | 3.95 | Transp., flat | Very | |
| | | | | | | | | | good | |
| R wt pea | 58 | 1.84 | 1% G | 98 | 0.6 | 9 × 9 | 3.88 | Transp., flat | Very | |
| | | | | | | | | | good | |
| Wt pea | 83 | 1.79 | 2% G | 98 | 1.1 | 8.8× | 3.88 | Transp., flat | Very | |
| | | | | | | | | | good | |
| WTpea 15/3/04- | 37 | 1.77 | H20 | 160 | — | 10.3 × 10.3 | 3.84 | Transp., flat | Good | |
| 1,2,3 | | | | | | | | | | |
| Initial | | | | | | | | | | |
| WT/18/03/04- | 57 | 1.77 | 1% G | 166 | 0.55 | 9.5 × 9.6 | 3.84 | Transp., slightly | Good | |
| 1,2,3 | | | | | | | | wrinkled | | |
| Initial | | | | | | | | | | |
| WT/24/3/04- | 37 | 1.77 | H20 | 166 | — | 10.2 × 10.2 | 3.84 | Milky, flat | Stable | |
| 1,2,3 | | | | | | | | | | |
| Initial | | | | | | | | | | |
| WT/16/04/04-1 | 66 | 2.11 | 1% G | 98 | 0.46 | 10.0 × 10.0 | 4.58 | Transp., flat | Very Good | |
| Initial | | | | | | | | | | |
| WT/16/04/04-2 | 66 | 2.11 | 1% G | 98 | 0.46 | 9.9 × 10.0 | 4.58 | Transp., flat | Very Good | |
| Initial | | | | | | | | | | |
| WT/16/04/04- | 74 | 2.53 | 1% G | 98 | 0.38 | 9.8 × 9.8 | 5.49 | Transp., flat | Very Good | |
| 3,4 | | | | | | | | | | |
| Initial | | | | | | | | | | |
| WT/16/04/04- | 84 | 3.37 | 1% G | 98 | 0.29 | 9.8 × 9.8 | 7.31 | Transp., flat, | Excellent | |
| 5,6 | | | | | | | | slightly | | |
| Initial | | | | | | | | wrinkled | | |
| WT/19/04/04-1 | | 5.05 | H20 | 98 | — | 9.6 × 9.6 | 9.58 | Transp., | Very Good | |
| Initial | | | | | | | | wrinkled | | |
| WT/19/04/04-2 | 48 | 5.05 | H20 | 98 | — | 9.8 × 9.8 | 7.15 | Transp., | Very Good | |
| Initial | | | | | | | | wrinkled, | | |
| | | | | | | | | cracked | | |
| WT/19/04/04- | 59 | 3.37 | H20 | 98 | — | 9.8 × 9.9 | 7.31 | Transp., | Very Good | |
| 3,4 | | | | | | | | wrinkled, | | |
| Initial | | | | | | | | cracked | | |
| WT/19/04/04- | 69 | 4.21 | H20 | 98 | — | 9.6 × 9.6 | 8.86 | Transp., not flat, | Very Good | |
| 5,6 | | | | | | | | brittle | | |
| Initial | | | | | | | | | | |
| WT/20/04/04B- | 100 | 2.53 | 3% G | 98 | 1.16 | 10.0 × 10.0 | 4.32 | Transp., flat | Very Good | |
| 1,2 | | | | | | | | | | |
| Initial | | | | | | | | | | |
| WT//20/04/04B- | 111 | 3.37 | 4% G | 98 | 1.14 | 10.0 × 10.0 | 5.79 | Transp., flat | Very good | |
| 3,4 | | | | | | | | | | |
| Initial | | | | | | | | | | |
| WT/98/20/04/04 | 172 | 4.21 | 5% G | 98 | 1.13 | 10.0 × 10.0 | 7.35 | Transp., flat | Very good | |
| B-5,6 | | | | | | | | | | |
| Initial | | | | | | | | | | |
| Rb/Fb002 | 43 | 1.76 | H2O | 98 | — | 10 × 10.1 | 3.82 | Transp, slightly | | |
| | | | | | | | | wrinkled | | |
| Rb/Fb004 | 60 | 1.76 | 1% G | 98 | 0.56 | 8.5 × 9.2 | 3.84 | Transp, slightly | | |
| | | | | | | | | wrinkled | | |
| Rb/Fb005 | 43 | 1.78 | H2O | 150 | — | | 3.90 | | | |
| rsim57/Fr158 | | 1.85 | H2O | 160 | — | | 4.01 | | | |
| rsim57/Fr183 | | 1.86 | 1% G | 160 | 0.53 | 9 × 9 | 4.05 | Milky | | |
| | | | | | | | | wrinkled | | |
| rug5/F5135 | | 1.92 | H2O | 120 | — | 10.3 × 10.3 | 4.18 | Transp, slightly | | |
| | | | | | | | | wrinkled | | |
| rug5/F5184 | 51 | 1.90 | 1% G | 120 | 0.52 | 9.2 × 9.4 | 4.18 | Slightly milky, | Very good | |
| | | | | | | 8.8 × 9 | | slightly | | |
| | | | | | | | | wrinkled | | |
| rug5/F5160 | 33 | 1.87 | H2O | 160 | — | | 4.27 | | | |
| rug5/F5185 | 50 | 1.85 | 1% G | 160 | 0.53 | | 4.05 | | | |
| Potato | | 0.42 | H2O | 98 | — | 10 × 10.1 | 0.95 | Transp. flat | Very | |
| | | | | | | | | | bad | |
| Potato | | 0.85 | H2O | 98 | — | 10.2 × 10.3 | 1.91 | Transp. flat | Very | |
| | | | | | | | | | bad | |
| Potato | 26 | 1.27 | H2O | 98 | — | 10.1 × 10.2 | 2.41 | Transp. flat | Very | |
| | | | | | | | | | bad | |
| Potato/Fpt102 | 58 | 1.27 | 1% | 98 | 0.78 | | 2.68 | Transp. Very | Very | |
| | | | | | | | | wrinkled | bad | |
| Potato/Fp135 | | 1.53 | H2O | 98 | — | | 2.60 | | | |
| Potato | | 1.53 | 2% G | 98 | 1.28 | 7.5 × 8.0 × 7C7.5 | 2.76 | Wrinkled | Very | |
| | | | | | | | | | bad | |
| Potato | | 1.53 | 3% G | 98 | 1.93 | 6.8 × 6.9 | 3.03 | Very wrinkled | Very bad | |
| | | | | | | 7 × 8.4 | | | | |
| Potato | | 1.53 | 1% G | 98 | 0.64 | | 2.64 | | | |
| Potato | | 1.78 | H2O | 98 | — | | 2.97 | | | |
| Potato | | 1.78 | 1% G | 98 | 0.55 | | 2.97 | | | |
| Potato | | 1.27 | H20 | | | ˜2 × 2 | ˜20 | | | |
| Waxy Maize | 36 | 1.82 | H2O | 98 | — | 10.4× | 3.97 | Transp, flat | Very bad | |
| (Amioca)/ | | | | | | | | | | |
| Fm170 or | | | | | | | | | | |
| Fm038 | | | | | | | | | | |
| Waxy Maize | 59 | 1.83 | 1% G | 98 | 0.54 | ˜8.5× | 3.97 | Transp., | Very | |
| (Amioca)/ | | | | | | | | very wrinkled | bad | |
| Fm120 | | | | | | | | | | |
| Wheat | 37 | 1.88 | H2O | 120 | — | | 4.11 | Milky | Very bad | 40, |
| (Sigma) | | | | | | | | flat | | 50 |
| Wheat | | 1.88 | H2O | 150 | — | | 4.08 | | | |
| Wheat | 35 | 1.88 | H2O | 160 | — | | 4.13 | Milky | Very bad | 42 |
| | | | | | | | | flat | | |
| Wheat | | 1.88 | 1% G | 120 | 0.52 | | 4.13 | Very wrinkled | Good | |
| | | 1.88 | | | | | | | | |
| Pea (Provital) | | 1.88 | H2O | 98 | — | 10 × 10 | 4.06 | | Good | |
| Pea (Provital) | | 1.88 | 1% G | 98 | 0.52 | 9 × 9 | 4.10 | | Very good | |
| WT/21/4/04-1,2, | 51 | 1.77 | 1% S | 98 | 0.55 | 10.0 × 10.0 | 3.84 | Transp., flat | Good | |
| Initial | | | | | | | | | | |
| WT/21/4/04A- | 75 | 1.77 | 2% S | 98 | 1.11 | 10.0 × 10.0 | 3.84 | Transp., flat | Good | |
| 3,4, | | | | | | | | | | |
| Initial | | | | | | | | | | |
| WT/21/4/04-5,6, | 106 | 1.77 | 3% S | 98 | 1.66 | 9.9 × 9.9 | 3.84 | Transp., flat, | Good | |
| Initial | | | | | | | | | | |
| Wt | 54 | 1.77 | 1% M | 98 | 0.55 | 10.3 × 10.3 | 3.84 | Transp., flat | Good | |
| 28/04/04-1,2 | | | | | | | | | | |
| Wt | 76 | 1.77 | 2% M | 98 | 1.11 | 9.8 × 9.9 | 3.84 | Transp., flat | Good | |
| 28/04/04-3,4 | | | | | | | | | | |
| Wheat | 36 | 1.88 | H20 | 166 | — | 9.9 × 9.9 | 4.08 | Transp., | Bad | |
| 25/03/04-1,2,3 | | | | | | | | wrinkled, | | |
| Initial | | | | | | | | cracked, | | |
| | | | | | | | | shattered | | |
| Wheat | 59 | 1.88 | 1% G | 166 | 0.52 | 9.9 × 9.9 | 4.08 | Transp., | Bad | |
| 26/03/04-1,2,3 | | | | | | | | wrinkled, weak, | | |
| Initial | | | | | | | | sticky | | |
| Chickpea | 37 | 1.89 | H20 | 98 | — | 10.0 × 10.0 | 4.12 | Transp., | Good | |
| 14/04/04A-1,2,3 | | | | | | | | wrinkled, | | |
| Initial | | | | | | | | shattered | | |
| C/P14/04/04B- | 64 | 1.89 | 1.2% G | 98 | 0.62 | 9.7 × 9.7 | 4.12 | Transp., | Very Good | |
| 1,2,3 | | | | | | | | wrinkled, | | |
| Initial | | | | | | | | not flat | | |
| Faba Bean | 36 | 1.88 | H20 | 98 | — | 10.2 × 10.2 | 4.11 | Transp., flat | Good | |
| 14/04/04A-4,5,6 | | | | | | | | | | |
| Initial | | | | | | | | | | |
| F/B14/04/04B- | 57 | 1.88 | 1.2% G | 98 | 0.63 | 9.9 × 9.9 | 4.10 | Transp., slightly | Very Good | |
| 4,5,6 | | | | | | | | wrinkled, | | |
| Initial | | | | | | | | | | |
| WT/15/4/04A- | 55 | 1.88 | 1% G | 98 | 0.55 | 10.2 × 10.2 | 3.84 | Transp., slightly | Good | |
| 1,2,3 | | | | 40° C. | | | | wrinkled | | |
| Initial | | | | | | | | | | |
| WT/15/4/04B- | 35 | 1.77 | H2O | 98 | — | 10.3 × 10.3 | 3.84 | Transp., | Good | |
| 1,2,3 | | | | 40° C. | | | | wrinkled | | |
| Initial | | | | | | | | | | |
| WT/20/4/04A- | 32 | 1.77 | H20 | 98 | — | 10.2 × 10.2 | 3.84 | Transp., | Good | |
| 1,2,3 | | | | 60° C. | | | | wrinkled | | |
| Initial | | | | | | | | | | |
| WT/20/4/04A- | 50 | 1.77 | 1% G | 98 | 0.55 | 10.3 × 10.3 | 3.86 | Transp., slightly | Very good | |
| 1,2,3 | | | | 60° C. | | | | wrinkled | | |
| Initial | | | | | | | | | | |

TABLE 4
Water Uptake by Films in Excess water (films were places in
water overnight)

| | Moisture content | Moisture content after |
|---|---|---|
| | When stored st 58% RH | holding in excess water |
| Film | (%) | (%) |
| 3% wt/3% G/98 | 50.6 | 68.4 |
| 20/4/04B-1 | | |
| 4% wt/4% G/98 | 45.0 | 66.4 |
| 20/4/04B-4 | | |

TABLE 5
A - Properties of films (equilibrated at 58% RH) prior to storage for 1 year

| | | | Polyol/ | Moisture | |
|---|---|---|---|---|---|
| | % | % | Starch | Content | Appearance & |
| Film | Polyol | Starch | Ratio | (%) | Properties |
| starch/ | 0.98 | 2.1 | 0.467 | 9.5 | Flat, transparent. |
| sorbitol | | | | | |
| starch/ | 1.96 | 2.1 | 0.933 | 10.3 | Flat, transparent & |
| sorbitol | | | | | quite flexible. |
| starch/ | 2.94 | 2.1 | 1.400 | 11.2 | Flat, transparent & |
| sorbitol | | | | | flexible. Slight |
| | | | | | precipitate |
| starch/ | 0.98 | 2.1 | 0.467 | 9.4 | Flat, transparent. |
| maltitol | | | | | |
| starch/ | 1.96 | 2.1 | 0.933 | 10.3 | Flat, transparent & |
| maltitol | | | | | flexible. |

B - Properties of films after 1 years storage at 58% RH

| | Polyol/Starch | |
|---|---|---|
| Film | ratio | Appearance & Properties after 1 year |
| starch/ | 0.467 | Flat, transparent. Small, discrete white areas of |
| sorbitol | | precipitation (5% surface). Flexible. |
| starch/ | 0.933 | Wrinkled. White powder (precipitation) |
| sorbitol | | covering 90% of film surface. Brittle. |
| starch/ | 1.4 | Wrinkled, shrunken. White powder |
| sorbitol | | (precipitation) covering 100% of film surface. |
| | | Brittle. |
| starch/ | 0.467 | Flat, transparent. Cloudy patches of precipitate |
| maltitol | | within film (10% surface). |
| starch/ | 0.933 | Flat. Cloudy patches of precipitate within film |
| maltitol | | (50% of surface). Brittle in these areas. |

TABLE 6

| | 3.41% Starch & | | |
|---|---|---|---|
| | 5.43% Glycerol | | |
| Storage | Force | Yield Stress | Elongation |
| (Month) | (N) | (MpA) | (%) |
| 1 | 0.835 | 0.691 | 24.9 |
| 2 | 0.988 | 0.795 | 32.4 |
| 3 | 1.095 | 0.890 | 31.9 |

TABLE 7
Effect of changing the glycerol concentration (glycerol/starch ratio)

| | | | | | Initial | Starch | Starch | |
|---|---|---|---|---|---|---|---|---|
| | | | Initial | Initial | Glycerol/ | Weight | Weight | Film |
| | Prep. | Drying | Starch | Glycerol | Starch | Per cast | Per Film | area |
| Film | Temp | Temp. | Conc. | Conc. | Ratio | area | Area | Cm2 |
| Code | (° C.) | (° C.) | (%) | (%) | (g/g) | (mg/cm²) | (mg/cm²) | (original = 106 cm²) |
| 13 | 98 | 20 | 3.4 | 0.00 | 0.00 | 7.3 | n.d. | n.d. |
| 14 | 98 | 20 | 3.4 | 1.0 | 0.3 | 7.3 | n.d | n.d. |
| 15 | 98 | 20 | 3.4 | 1.9 | 0.6 | 8. | 9.4 | 90 |
| 16 + 42 | 98 | 20 | 3.4 | 3.7 | 1.1 | 8.1 | 8.3 | 104 |
| 17 + 18 + 20 | 98 | 20 | 3.4 | 5.4 | 1.6 | 8.1 | 8.3 | 103 |
| 25 | 98 | 20 | 3.4 | 7.1 | 2.1 | 8.1 | 8.4 | 102 |
| 26 | 98 | 20 | 3.4 | 8.7 | 2.6 | 8.1 | 8.9 | 96 |
| 27 | 98 | 20 | 3.4 | 10.3 | 3.0 | 8.2 | 9.3 | 94 |
| 28 | 98 | 20 | 3.4 | 11.8 | 3.5 | 8.1 | 9.9 | 87 |
| 29 | 98 | 20 | 3.4 | 13.2 | 3.9 | 8.1 | 9.2 | 93 |
| 30 | 98 | 20 | 3.4 | 14.6 | 4.3 | 8.0 | 9.0 | 94 |
| 31 | 98 | 20 | 3.4 | 16.0 | 4.7 | 8.1 | 9.0 | 94 |
| 33 | 98 | 20 | 3.4 | 17.3 | 5.1 | 8.1 | n.d | n.d. |
| 34 | 98 | 20 | 3.4 | 19.8 | 5.8 | 8.1 | n.d. | n.d. |
| 35 | 98 | 20 | 3.4 | 23.3 | 6.8 | 8.1 | 8.6 | 100 |

| | Av. | | | | | |
|---|---|---|---|---|---|---|
| | Thickness | | Max | Yield | | |
| Film | of Film | | Force | stress | Elongation | Water |
| Code | (μm) | Appearance | (N) | (MPa) | (%) | cont % |
| 13 | 67 | Flat, | 16* | 35* | 2.1* | 11 |
| | | transparent | | | | |
| 14 | 85 | Flat, | 16* | 38* | 2.6* | 9 |
| | | transparent | | | | |
| 15 | 108 | Flat, | 6.5 | 8.73 | 20 | 12 |
| | | transparent | | | | |
| 16 + 42 | 105 | Flat, | 1.8 | 1.9 | 37 | 13 |
| | | transparent | | | | |
| 17 + 18 + 20 | 174 | Flat, | 0.9 | 0.8 | 32 | 13 |
| | | transparent | | | | |
| 25 | 216 | Flat, | 0.7 | 0.5 | 28 | 14 |
| | | transparent | | | | |
| 26 | 250 | Flat, | 0.4 | 0.2 | 33 | 13 |
| | | transparent | | | | |
| 27 | 296 | Flat, | 0.4 | 0.2 | 47 | 12 |
| | | transparent | | | | |
| 28 | 320 | Flat, | 0.3 | 0.1 | 34 | 12 |
| | | transparent, | | | | |
| | | felt very | | | | |
| | | moist. | | | | |
| 29 | 378 | Flat, | 0.2 | 0.1 | 29 | 12 |
| | | transparent, | | | | |
| | | felt very | | | | |
| | | moist. | | | | |
| 30 | 377 | Flat, | 0.2 | 0.1 | 36 | 14 |
| | | transparent, | | | | |
| | | felt very | | | | |
| | | moist. | | | | |
| 31 | 400 | Flat, | 0.2 | 0.1 | 32 | 15 |
| | | transparent, | | | | |
| | | felt very | | | | |
| | | moist. | | | | |
| 33 | 456 | Flat, | 0.1 | 0.04 | 33 | 15 |
| | | transparent, | | | | |
| | | felt very | | | | |
| | | moist. | | | | |
| 34 | 495 | Flat, less | 0.004 | 0.01 | 14 | 15 |
| | | transparent, | | | | |
| | | moist, gel- | | | | |
| | | like. | | | | |
| 35 | 533 | Flat, | n.d. | n.d. | n.d. | n.d. |
| | | opaque, gel- | | | | |
| | | fell apart | | | | |
| | | on handling | | | | |

n.d. = not determined

TABLE 8
Effect of changing the weight of starch per film (mg/cm²)

| | | | | | Initial- | | | | | | | | | |
|---|---|---|---|---|---|---|---|---|---|---|---|---|---|---|
| | | | | Initial | Glyc- | Starch | | Film | | | | | | |
| | | | Initial | Glyc- | erol/ | Weight | Starch | area | | | | | | |
| | Prep. | Drying | Starch | erol | Starch | Per Cast | Weight | (cm²) | Thickness | | Max | Yield | | Water |
| Film | Temp | Temp. | Conc. | Conc. | Ratio | Area | Per Film | (original- | of Film | | Force | Stress | Elongation | cont |
| Code | (° C.) | (° C.) | (%) | (%) | (g/g) | (mg/cm²) | (mg/cm²) | 106 cm²) | (μm) | Appearance | (N) | (MPa) | (%) | (%) |
| 19 | 98 | 20 | 3.4 | 5.4 | 1.6 | 5.8 | 5.9 | 104 | 122 | Flat, | 0.8 | 0.9 | 38 | 13 |
| | | | | | | | | | | transparent | | | | |
| 18 + | 98 | 20 | 3.4 | 5.4 | 1.6 | 8.1 | 8.4 | 102 | 171 | Flat, | 0.9 | 0.8 | 32 | 13 |
| 20 | | | | | | | | | | transparent | | | | |
| 21 | 98 | 20 | 3.4 | 5.4 | 1.6 | 12.0 | 12.5 | 102 | 243 | Flat, | 1.2 | 0.7 | 33 | 13 |
| | | | | | | | | | | transparent | | | | |
| 22 | 98 | 20 | 3.4 | 5.4 | 1.6 | 16.2 | 24.2 | 71 | 589 | Flat, | 1.4 | 0.3 | 25 | 13 |
| | | | | | | | | | | transparent | | | | |
| 23 + | 98 | 20 | 3.4 | 5.4 | 1.6 | 24.1 | 41.2 | 62 | 962 | Flat, | 1 | 0.15 | 10 | 14 |
| 24 | | | | | | | | | | transparent | | | | |

TABLE 9
Effect of drying temperature on film properties

| | | | | | Initial | | | | | Starch | | | |
|---|---|---|---|---|---|---|---|---|---|---|---|---|---|
| | | | Initial | Initial | Glycerol/ | | | | | Weight | Starch | | |
| | Prep. | Drying | Starch | Glycerol | Starch | Moisture | Max | Yield | | Per cast | Weight | Thickness of | |
| Film | Temp | Temp. | Conc. | Conc. | Ratio | Content | Force | Stress | Elongation | Area | Per Film | Film | |
| Code | (° C.) | (° C.) | (%) | (%) | (g/g) | (%) | (N) | (MPa) | (%) | (mg/cm2) | (mg/cm²) | (μm) | Appearance |
| 17 | 98 | 20 | 3.4 | 5.4 | 1.6 | 11 | 0.8 | 0.7 | 25 | 8.2 | 8.4 | 179 | Flat, |
| | | | | | | | | | | | | | transparent |
| 17a | 98 | 60 | 3.4 | 5.4 | 1.6 | 13 | 1.4 | 1.3 | 37 | 8.2 | 8.4 | 148 | Flat, |
| | | | | | | | | | | | | | transparent |

TABLE 10
Antibacterial testing of films

| Type of Film | Initial count | 24 hour count |
|---|---|---|
| Inoculum | 1 × 10⁵ | 5.8 × 10⁵ |
| Starch/glycerol film | 1 × 10⁵ | 4.1 × 10³ |
| Starch/glycerol + Iodine | 1 × 10⁵ | <1 × 10² |
| film | | |

TABLE 11
Effect of changing starch concentration (%) and weight per Film and per Cast Area (mg/cm²)

| | | | | | Initial | | | | | | | | | |
|---|---|---|---|---|---|---|---|---|---|---|---|---|---|---|
| | | | | Initial- | Glyc- | Starch | Starch | | | | | | | |
| | | | Initial | Glyc- | erol/ | Weight | Weight | Film area | Thick- | | | | | |
| | Prep. | Drying | Starch | erol | Starch | Per Cast | Per Film | (cm²) | ness | | Max | Yield | Elon- | Water |
| Film | Temp | Temp. | Conc. | Conc. | Ratio | Area | Area | (Original = | of Film | | Force | Stress | gation | Cont |
| Code | (° C.) | (° C.) | (%) | (%) | (g/g) | (mg/cm²) | (mg/cm²) | 106) | (μm) | Appearance | (N) | (MPa) | (%) | (%) |
| 1 | 98 | 20 | 1.8 | 0 | 0 | 4.2 | 4.2 | 106 | 33 | Flat, | 11.2 | 45.4 | 3 | 8 |
| | | | | | | | | | | transparent | | | | |
| 10 | 98 | 20 | 2.1 | 1.0 | 0.5 | 4.5 | n/d | n/d | 60 | Flat, | 10* | 15* | 3* | n/d |
| | | | | | | | | | | transparent | | | | |
| 2 | 98 | 20 | 1.8 | 1.9 | 1.1 | 4.2 | 5.1 | 87 | 66 | Flat, | 1.2 | 2.6 | 30 | 9 |
| | | | | | | | | | | transparent | | | | |
| 3 | 98 | 20 | 1.8 | 2.9 | 1.6 | 4.2 | 5.2 | 85 | 87 | Fat, | 0.5 | 0.9 | 24 | 11 |
| | | | | | | | | | | transparent | | | | |
| 11 | 98 | 20 | 2.5 | 1 | 0.4 | 5.5 | n/d | n/d | 69 | Flat, | 11* | 23* | 2* | n/d |
| | | | | | | | | | | transparent | | | | |
| 12 | 98 | 98 | 2.5 | 2.9 | 1.2 | 4.3 | n/d | n/d | 89 | Flat, | 2 | 3 | 28 | 11 |
| | | | | | | | | | | transparent | | | | |
| 36 | 98 | 20 | 5.1 | 0 | 0 | 9.2 | 10.8 | 90 | 87 | Rippled, | n.d. | n.d. | n.d. | n.d. |
| | | | | | | | | | | transparent | | | | |
| 37 | 98 | 20 | 5.1 | 4.8 | 0.9 | 11.9 | 13.6 | 93 | 200 | Flat, | 2.9 | 2.2 | 33 | 13 |
| | | | | | | | | | | transparent | | | | |
| 38 | 98 | 20 | 5.1 | 7.2 | 1.4 | 11.9 | 14.5 | 87 | 264 | Flat, | 1.4 | 0.8 | 21 | 16 |
| | | | | | | | | | | transparent | | | | |
| 39 | 98 | 20 | 6.0 | 0 | 0 | 10.5 | 12.1 | 92 | 53 | Rippled, | n/d | n.d. | n.d. | 9 |
| | | | | | | | | | | transparent, | | | | |
| | | | | | | | | | | initial | | | | |
| | | | | | | | | | | solution | | | | |
| | | | | | | | | | | very viscous | | | | |
| 40 | 98 | 20 | 6.0 | 5.8 | 1 | 6.7 | 7.5 | 95 | 150 | Rippled, | n/d | n.d. | n.d. | 13 |
| | | | | | | | | | | transparent, | | | | |
| | | | | | | | | | | initial | | | | |
| | | | | | | | | | | solution | | | | |
| | | | | | | | | | | very viscous | | | | |
| 41 | 98 | 20 | 6.0 | 8.6 | 1.4 | 6.4 | 7.5 | 90 | 192 | Rippled, | n/d | n.d. | n.d. | 13 |
| | | | | | | | | | | transparent, | | | | |
| | | | | | | | | | | initial | | | | |
| | | | | | | | | | | solution | | | | |
| | | | | | | | | | | very viscous | | | | |

n.d. = not determined.
Max force, Yield stress and Elongation determined at 3.0 mm/min except for * = 0.6 mm/min.

TABLE 12
Amount of water absorbed by films

| | | | Initial | Strach | | |
|---|---|---|---|---|---|---|
| | | Initial | Glycerol/ | Weight | Absorb- | Absorb- |
| | Initial | Glycerol | Starch | Per | ency | ency |
| Film | Starch | Conc. | Ratio | Cast Area | of Film | of Film |
| Code | Conc. (%) | (%) | (g/g) | (mg/cm²) | mg/cm² | g/g |
| 1 | 1.77 | 0.00 | 0.00 | 4.2 | 12.0 | 2.2 |
| 2 | 1.77 | 1.92 | 1.09 | 4.2 | 6.4 | 0.7 |
| 3 | 1.77 | 2.88 | 1.63 | 4.2 | 3.2 | 0.4 |
| 7 | 1.77 | 0.96 | 0.54 | 4.0 | 9.9 | 2.7 |
| 12 | 2.53 | 2.88 | 1.14 | 4.3 | 8.0 | 0.8 |
| 13 | 3.37 | 0.00 | 0.00 | 7.3 | 12.4 | 1.6 |
| 14 | 3.37 | 0.96 | 0.29 | 7.3 | 11.0 | 1.3 |
| 15 | 3.41 | 1.92 | 0.56 | 8.0 | 13.3 | 0.9 |
| 16 | 3.41 | 3.69 | 0.56 | 8.1 | 9.1 | 0.6 |
| 17a | 3.41 | 5.43 | 1.59 | 8.2 | 10.7 | 0.5 |
| 19 | 3.41 | 5.43 | 1.59 | 5.8 | 8.9 | 0.4 |
| 20 | 3.41 | 5.43 | 1.59 | 8.1 | 9.6 | 0.5 |
| 21 | 3.41 | 5.43 | 1.59 | 12.0 | 11.9 | 0.3 |
| 22 | 3.41 | 5.43 | 1.59 | 16.2 | 18.7 | 0.3 |
| 23/24 | 3.41 | 5.43 | 1.59 | 24.1 | 35.5 | 0.3 |
| 26 | 3.41 | 8.73 | 2.56 | 8.1 | 6.6 | 0.2 |
| 29 | 3.41 | 13.24 | 3.88 | 8.1 | 6.9 | 0.1 |
| 36 | 5.0 | 0.00 | 0.00 | 9.2 | 18.5 | 1.8 |
| 37 | 5.0 | 4.80 | 1.14 | 11.9 | 17.1 | 0.5 |
| 38 | 5.0 | 7.20 | 1.71 | 11.9 | 8.1 | 0.3 |
| 39 | 6.0 | 0.00 | 0.00 | 10.5 | 13.6 | 2.4 |
| 40 | 6.0 | 5.76 | 1.14 | 6.7 | 6.5 | 0.4 |
| 41 | 6.0 | 8.64 | 1.71 | 6.4 | 6.6 | 0.5 |

TABLE 13
Characterisation of films made with inserted membranes

| | | | | | Initial | Starch | | | |
|---|---|---|---|---|---|---|---|---|---|
| | | | Initial | Initial | Glycerol/ | Weight | | | |
| | Prep. | Drying | Starch | Glycerol | Starch | Per Cast | Film Size | Thickness | Incorporation |
| | Temp | Temp | Conc. | Conc. | Ratio | Area | (original = | of Film | of |
| Insert Type | (° C.) | (° C.) | (%) | (%) | (g/g) | (mg/cm²) | 10.3 cm × 10.3 cm) | (μm) | Insert |
| Nylon Gauze - | 98 | 20 | 3.41 | 5.43 | 1.59 | 8.3 | 10.2 × 10.2 | 812 | Excellent |
| submerged | | | | | | | | | |
| Nylon Gauze - over | 98 | 20 | 3.41 | 5.43 | 1.59 | 8.4 | 10.1 × 10.2 | 724 | Very good |
| laid | | | | | | | | | |
| Nylon - submerged | 98 | 20 | 3.41 | 5.43 | 1.59 | 8.3 | 10.2 × 10.2 | 262 | Excellent |
| Nylon - over laid | 98 | 20 | 3.41 | 5.43 | 1.59 | 8.2 | 10.3 × 10.3 | 261 | Very good |
| Bandage - | 98 | 20 | 3.41 | 5.43 | 1.59 | 9.7 | 9.7 × 9.7 | 674 | Poor |
| submerged | | | | | | | | | |
| Bandage - over laid | 98 | 20 | 3.41 | 5.43 | 1.59 | 9.9 | 10.1 × 10.1 | 662 | Poor |
| Rayon/polyester | 98 | 20 | 3.41 | 5.43 | 1.59 | 8.2 | 10.3 × 10.3 | 354 | Excellent |
| (Miracloth) - | | | | | | | | | |
| submerged | | | | | | | | | |
| Rayon/polyester | 98 | 20 | 3.41 | 5.43 | 1.59 | 8.2 | 10.3 × 10.3 | 369 | Excellent |
| (Miracloth) - over | | | | | | | | | |
| laid | | | | | | | | | |
| Natural Gauze - | 98 | 20 | 3.41 | 5.43 | 1.59 | 8.3 | 10.3 × 10.3 | 515 | Excellent |
| submerged | | | | | | | | | |
| Natural Gauze - | 98 | 20 | 3.41 | 5.43 | 1.59 | 8.1 | 10.3 × 10.3 | 550 | Excellent |
| over laid | | | | | | | | | |

TABLE 14
Effect of inserting membranes into the films on mechanical properties and water absorption

| | | | | | Initial | | | | Starch | | |
|---|---|---|---|---|---|---|---|---|---|---|---|
| | | | Initial | Initial | Glycerol/ | | | | Weight | | |
| | Prep. | Drying | Starch | Glycerol | Starch | Max | Tensile | | Per Cast | Absorbency | |
| | Temp | Temp. | Conc. | Conc. | Ratio | Force | Strength | Elongation | Area | of Film | Absorbency |
| | (° C.) | (° C.) | (%) | (%) | (g/g) | (N) | (MPa) | (%) | (mg/cm²) | mg/cm² | of Film g/g |
| No inserts | 98 | 20 | 3.41 | 5.43 | 1.59 | 0.9 | 0.8 | 31 | 8.1 | 10 | 0.53 |
| Nylon Gauze - | 98 | 20 | 3.41 | 5.43 | 1.59 | 1.7 | 0.3 | 25 | 8.3 | 48 | 1.34 |
| submerged | | | | | | | | | | | |
| Nylon - | 98 | 20 | 3.41 | 5.43 | 1.59 | 29.2 | 15.8 | 16 | 8.25 | 13 | 0.45 |
| submerged | | | | | | | | | | | |
| Bandage - | 98 | 20 | 3.41 | 5.43 | 1.59 | 2.9 | 0.6 | 11 | 9.7 | n.d. | n.d. |
| submerged | | | | | | | | | | | |
| Rayon/polyester | 98 | 20 | 3.41 | 5.43 | 1.59 | 9.4 | 3.8 | 14 | 8.2 | n.d. | n.d. |
| (Miracloth) - | | | | | | | | | | | |
| submerged | | | | | | | | | | | |
| Rayon/polyester | 98 | 20 | 3.41 | 5.43 | 1.59 | n.d. | n.d. | n.d. | 8.2 | 23 | 0.92 |
| (Miracloth) - | | | | | | | | | | | |
| Over laid | | | | | | | | | | | |
| Natural Gauze - | 98 | 20 | 3.41 | 5.43 | 1.59 | n.d. | n.d. | n.d. | 8.3 | 26 | 0.73 |
| submerged | | | | | | | | | | | |

TABLE 15
Final composition of films

| | | | Initial | Initial | Initial | St/cast | Final | | | |
|---|---|---|---|---|---|---|---|---|---|---|
| | H | D | St | Gly | Gly/St | area | Gly/St | Final | Final | Final |
| | TC | TC | conc, % | conc % | g/g | mg/cm² | g/g | Gly % | St % | H₂O % |
| 2 | 98 | 20 | 1.8 | 1.9 | 1.1 | 4.2 | 0.6 | 35 | 56 | 9 |
| 14 | 98 | 20 | 3.4 | 1 | 0.3 | 7.3 | 0.15 | 12 | 79 | 9 |
| 15 | 98 | 20 | 3.4 | 1.9 | 0.6 | 8.0 | 0.35 | 23 | 65 | 12 |
| 42 | 98 | 20 | 3.4 | 3.8 | 1.1 | 8.2 | 1* | 45* | 45* | 10 |
| 19 | 98 | 20 | 3.4 | 5.4 | 1.6 | 5.8 | 1.5 | 53 | 36 | 11 |
| 18-20 | 98 | 20 | 3.4 | 5.4 | 1.6 | 8.2 | 1.5 | 52 | 35 | 13 |
| 21 | 98 | 20 | 3.4 | 5.4 | 1.6 | 11.9 | 1.5 | 53 | 35 | 13 |
| 22 | 98 | 20 | 3.4 | 5.4 | 1.6 | 16.2 | 1.56 | 53 | 34 | 13 |
| 24 | 98 | 20 | 3.4 | 5.4 | 1.6 | 24.3 | 1.5 | 52 | 34 | 14 |
| 25 | 98 | 20 | 3.4 | 7.1 | 2.1 | 8.1 | 2.1 | 58 | 28 | 14 |
| 26 | 98 | 20 | 3.4 | 8.7 | 2.6 | 8.1 | 2.4 | 61 | 25 | 14 |
| 27 | 98 | 20 | 3.4 | 10.3 | 3.0 | 8.1 | 2.8 | 64 | 23 | 13 |
| 28 | 98 | 20 | 3.4 | 11.8 | 3.5 | 8.2 | 3.0 | 65 | 22 | 14 |
| 29 | 98 | 20 | 3.4 | 13.2 | 3.9 | 8.1 | 3.4 | 67 | 20 | 14 |
| 30 | 98 | 20 | 3.4 | 14.6 | 4.3 | 8 | 3.5 | 67 | 19 | 14 |
| 31 | 98 | 20 | 3.4 | 16.0 | 4.7 | 8.1 | 3.5 | 66 | 19 | 15 |
| 33 | 98 | 20 | 3.4 | 17.3 | 5.1 | 8.1 | 4.5 | 68 | 15 | 16 |
| 34 | 98 | 20 | 3.4 | 19.8 | 5.8 | 8.1 | 5.0 | 70 | 14 | 16 |
| 35 | 98 | 20 | 3.4 | 23.3 | 6.8 | 8.1 | 6.3 | 69 | 11 | 20 |
| 37 | 98 | 20 | 4.2 | 4.8 | 1.14 | 11.9 | 1.03* | 44* | 43* | 13 |
| 39 | 98 | 20 | 4.2 | 7.2 | 1.7 | 11.9 | 1.5* | 50* | 33* | 16.5 |
| 40 | 98 | 20 | 5.1 | 5.8 | 1.14 | 6.7 | 1.0* | 43* | 43* | 14 |
| 41 | 98 | 20 | 5.1 | 8.64 | 1.7 | 5.4 | 1.5 | 52* | 34* | 14 |

*approximations based on a coefficient of 0.9

TABLE 16
Final composition of starch films without glycerol

| | Starch | | | | | | |
|---|---|---|---|---|---|---|---|
| | conc | | | | | | |
| Source | in | Heating | Starch/cast | Drying | | | Composition |
| of pea | initial | Temp | area | Temp | Water in | Starch in | method |
| starch | susp, % | ° C. | (mg/cm²) | ° C. | film, % | film, % | used |
| rug4 | 2.7 | 98 | 6.6 | 20 | 15 | 85 | 1 |
| rug4 | 0.5 | 98 | 1.1 | 20 | 10 | 90 | 1 |
| rug4 | 1.9 | 120/160 | 4.2/4.0 | 20 | 13/13 | 87/87 | 1 |
| WT | 1.8 | 98 | 3.9 | 20 | 13/14 | 87/86 | 1 |
| WT | 5.05 | 98 | 7.2/9.6/10.5 | 20 | 12/12/9 | 88/88/91 | 1/1/2 |
| WT | 3.4 | 98 | 7.3 | 20 | 13 | 87 | 1 |
| WT | 4.2 | 98 | 8.9/9.2 | 20 | 13/10 | 87/90 | 1/2 |
| WT | 1.8 | 98 | 4.2 | 20 | 9/8 | 91/92 | 1/2 |
| WT | 1.8 | 120/160/ | 3.8/3.9/3.8 | 20 | 13/13/12 | 87/87/88 | 2 |
| | | 166 | | | | | |
| WT | 3.4 | 98 | 7.3 | 20 | 11 | 89 | 2 |

TABLE 17
A. Moisture vapour transmission rate (MVTR) and
absorbency properties of starch films containing glycerol

| Film | | mg | MVTR | Absorbency |
|---|---|---|---|---|
| code | Film Composition | starch/cm² | (g/10 cm²/24 h) | (g/10 cm²/24 h) |
| 16 | 3.41% st & 3.69% | 8.1 | 2.63 | 0.11 |
| | gly, 1.1 gly/st, g/g | | | |
| 18 | 3.41% st & 5.43% | 8.1 | 2.77 | 0.09 |
| | gly, 1.6 | | | |
| 27 | 3.41% st & 10.29% | 8.1 | 2.90 | 0.09 |

B. MVTR comparison with existing film dressings

| Hydrocolloid Wound Dressing | MVTR (g/10 cm²/48 hours) |
|---|---|
| Tegasorb Thin | 1.53 |
| Tegasorb | 2.12 |
| Cutinova Hydro | 1.85 |
| Askina Biofilm Transparent | 0.51 |
| Askina Transorbent | 5.12 |
| Comfeel Plus Plaques Biseautees | 1.97 |
| Comfeel Plus Transparenter | 2.76 |
| Comfeel Plus Flexibler (sample 1) | 1.33 |
| Comfeel Plus Flexibler (sample 2) | 1.75 |
| Varihesive E | 0.17 |
| Granuflex | 0.15 |
| Hydrocoll | 1.05 |
| Algoplaque | 0.25 |

1. A process for producing a starch film comprising the steps of:
(i) providing a preparation of starch from C-type starch granules,
(ii) heating and agitating the starch as a suspension in water and a polyol to form a starch paste,
(iii) cooling and drying the starch paste to form a starch film,
wherein said starch film has a cast starch/unit area range of between 1 to 45 mg/cm²

2. A process as claimed in claim 1 wherein the starch granules contain a proportion of A-polymorphs of between 10 and 90%.

3. A process as claimed in claim 1 wherein the starch granules contain an amylose content of between 5 and 75%.

4. A process as claimed in claim 1 wherein the starch granules contain total crystallinity, when equilibrated at 40-60% RH, of between 10 and 30%.

5. A process as claimed in claim 1 wherein the starch granules contain a proportion of double helices of between 15 and 55%.

6. A process as claimed in claim 1 wherein the starch granules are obtained from legume seeds.

7-8. (canceled)

9. A process as claimed in claim 6 wherein the C-type starch is selected from those described in Table 1.

10-20. (canceled)

21. A starch film obtained or obtainable by a process of claim 1.

22. A starch film as claimed in claim 21 which has a polyol/starch ratio of between 0.5 and 6.

23. A starch film as claimed in claim 22 which comprises between 15 and 70% glycerol.

24-31. (canceled)

32. Use of a starch film as a wound dressing.

33. Use of a starch film for the control, growth inhibition, or destruction of a microorganism.

34-35. (canceled)

36. Use as claimed in claim 33 wherein the starch film is applied to a surface on which it is desired to control the microorganism.

37. Use as claimed in claim 36 wherein the surface is on a human or animal body.

38. Use as claimed in claim 32 wherein the starch film comprises between 5 and 55% water.

39. Use as claimed in claim 32 wherein the starch film comprises between 10 and 95% starch on a dry weight basis.

40. Use as claimed in claim 32 wherein the starch film is capable of % elongation of less than 40.

41. Use as claimed in claim 32 wherein the starch film has a yield stress of 0.04 to 50 MPA.

42. Use as claimed in claim 32 wherein the starch film consists essentially of starch and water and optionally a polyol.

43-46. (canceled)

47. Use as claimed in claim 32 in a method of treating or promoting healing of a wound, which method comprises contacting the wound with the film, or adhering the film to the skin of a patient in need of the same.

48. (canceled)

49. A wound-dressing material or article comprising:
(i) a starch film, and
(ii) a support layer.

50. A wound-dressing material or article as was claimed in claim 49 wherein the support layer is integral with the starch film.

51. A wound-dressing material or article as claimed in claim 49 wherein the support layer is a natural or synthetic polymer.

52. A wound-dressing material or article as claimed in claim 49 wherein the starch film comprises 5-55% water.

53. A wound-dressing material or article as claimed in claim 49 wherein the starch film comprises 10-95% starch on a dry weight basis.

54. A wound-dressing material or article as claimed in claim 49 wherein the starch film is capable of % elongation of less than 40.

55-56. (canceled)

57. A wound-dressing material or article as claimed in claim 49 wherein the starch film consists essentially of starch, water and glycerol.

58-61. (canceled)

62. A starch film comprising active ingredients selected from: antibodies; antibiotic agents; antiseptic agents; anti-inflammatory agents.

63. A starch film as claimed in claim 62 wherein the starch film consists essentially of starch and water and optionally a polyol.

64. A starch film as claimed in claim 21 comprising active ingredients selected from: antibodies; antibiotic agents; antiseptic agents: anti-inflammatory agents.
